(19) United States
(12) Patent Application Publication
(10) Pub. No.: US 2004/0158893 A1
(43) Pub. Date: Aug. 12, 2004

(54) Final segregation of male meiotic products in plants

(76) Inventors: Hugh Dickinson, Oxford (GB); Roderick Scott, Bath (GB); Melissa Speilman, Oxford (GB)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/473,276

(22) PCT Filed: Mar. 28, 2002
(86) PCT No.: PCT/GB02/01466

(30) Foreign Application Priority Data
Mar. 30, 2001 (GB) 0108050.6

(51) Int. Cl. A01H 1/00; C12N 15/82

(52) U.S. Cl. 800/287

(57) ABSTRACT

The invention provides a plant in which, by virtue of modulation of the expression of a TETRASPORE (TES) or TES-like (TLK) gene, or of modulation of the activity of a protein encoded by such a gene, tetrad formation in the anther is disrupted, or is capable of being disrupted, such that callose cross-wall formation in the tetrad fails to the extent that fusion between two or more of the four microspore nuclei may occur.

### FIELD OF THE INVENTION

The present invention is in the field of plant biotechnology. More specifically, it pertains to techniques by which crosses between plants of different taxa are made possible. This is made possible by the cloning and characterisation of the TETRASPORE (TES) gene, whose sequence is provided herein.

### BACKGROUND OF THE INVENTION

Plant Genome Ploidy

Plants differ from animals in that they exhibit a range of genome ploidy. Animal somatic cells are usually diploid, and animal gametes haploid. Whilst many plants also conform to this, others are, for example, tetraploid (and thus their gametes are diploid) or hexaploid (and thus their gametes are triploid). For example, wheat is generally hexaploid. In some cases, genome ploidy varies within a single species, e.g. in certain ornamental plants, and also within the model species Arabidopsis.

This poses an important problem for plant breeders. It is difficult to perform crosses between plants of different genome ploidies and most such crosses fail. In some cases, it is possible to alter genome ploidy by tissue culture. Normally, small explants (pieces of leaf or stem) are cultured in vitro in the presence of colchicine which inhibits the mitotic spindle. The cells then undergo another round of DNA synthesis without cell division. The colchicine is then removed from the culture medium allowing the cells to progress into cell division. With suitable care and attention, it is possible to generate tetraploid and hexaploid plants by this method. However, this is extremely time-consuming, as it involves generating a parent plant with the desired parental characteristics but altered somatic cell ploidy.

Genome Ploidy in Pollen Development and Fertilisation in Plants

In plants, pollen carries the male gametes (sperm) to the female ones (ova). In the anther of the male parent plant, pollen development begins with the formation of a tetrad of microspores by meiosis. These are the progenitors of the male gametic cells. In, for example, a diploid plant, the somatic cells are diploid and the microspores are haploid. In the normal tetrad, the four microspores are separated from one another by callose cross-walls. Each microspore then undergoes a further series of mitotic divisions, leading to a male gamete cell containing two sperm nuclei and a vegetative nucleus. All of these are haploid. Pollen containing such cells is released from the male parent plant and fertilises the female parent plant.

During this process, one of the sperm nuclei fuses with the haploid ovum to form a diploid cell generally equivalent to an animal zygote. However, the other sperm nucleus fuses with a diploid “central cell” of the female parent plant to form a triploid cell. This is not a feature of the reproductive process in animals. Divisions of the triploid cell generate the endosperm, which provides resources for the growing embryo in the developing seed.

In the above, it has been assumed that the plant has diploid (2n) somatic cells, thus haploid (1n) gametes, diploid central cells and a triploid (3n) endosperm. In tetraploid (4n) systems, the gametes are diploid, the central cells tetraploid and the endosperm hexaploid. In hexaploid systems, the gametes are triploid, the central cells hexaploid and the endosperm nonaploid (9n).

It can therefore be seen that the normal ratio of genome contributions in the endosperm is 2 maternal: 1 paternal. In most plants, Arabidopsis being something of an exception, this “endosperm balance number” is crucial to successful seed development. This is why crosses between plants of different genome ploidies generally fail.

Clearly, it would be beneficial to the plant breeding industry to have available more straightforward methods for generating genomes of a range of different ploidies in the gametes of parent plants.

### SUMMARY OF THE INVENTION

We have cloned the TETRASPORE (TES) gene of Arabidopsis. Its sequence is provided herein.

We have previously determined that the product of the TES gene is required for male meiotic cytokinesis (Spielman et al, Development 124, 2645-2657 (1997)). In that paper, we described four mutant alleles of the TES gene and noted that, following failure of male meiotic cytokinesis in the mutants, all four microspore nuclei remain within the same cytoplasm, with some completing their developmental programmes to form functional pollen nuclei. The meiotic division seen in normal pollen development takes place in TES gene mutants. In other words, the TES gene appears to be necessary for the formation of the callose cross-walls in the tetrad that normally separate the four microspore nuclei. We have also previously investigated parent-of-origin effects on seed development in Arabidopsis (Scott et al, Development 125, 3329-3341 (1998)).

We have now cloned the TES gene and investigated its properties. As discussed above, tetrads in TES gene mutants lack the callose cross-walls that normally separate the four mircospore nuclei. The result of this is that very large pollen grains containing all the products of meiosis are formed. Strikingly, these meiotic products go on to develop relatively normally to form sperms and vegetative cells. However, we have now determined that fusion sometimes takes place between the microspore nuclei resulting in the formation of “male germ units” of differing ploidy. As discussed above, a male sperm nucleus would normally be haploid in a diploid plant, diploid in a tetraploid plant and triploid in a hexaploid plant. However, in TES gene mutant plants, sperm nuclei with higher ploidy are sometimes formed. Importantly, the ploidy of male germ units in TES gene mutants is also heterogeneous such that, for example, some may be haploid, some diploid, some triploid and some tetraploid in Arabidopsis.

This has important implications in plant breeding, because it will facilitate wide crosses within and between species wherein the male and female parent plants have different genome ploidies. Essentially, because of the variation in the ploidy of the sperm generated in plants in which the product of the TES gene is suppressed or inactivated, there is a good chance that the ploidy of some of the sperm will conform to the ploidy of the female parent plant, thus ensuring correct balance numbers for the zygote and, crucially, the endosperm of the resultant embryo. This offers a much more convenient way of performing mixed-ploidy crosses than is currently available.

Significantly, TES gene mutant plants (tes) also seem to be unaffected in the female development, so there is no reason to believe that manipulating the TES gene will compromise female fertility. TES gene mutants show reduced fertility, presumably because of the overall lack of congruence with the ploidy of the female parent plant. However, this is substantially outweighed by the advantage of being able to generate a range of sperm ploidies to facilitate crossing. In addition, when TES protein activity is suppressed, e.g. by the use of antisense or RNAi techniques, such suppression can be rendered inducible so that the variability of sperm ploidy can be switched on and off as desired.

The TES gene has been identified by positional cloning. The TES genomic sequence extends for some 10 Kb, and the cDNA encodes a protein of 932 amino acids. Importantly, sequence comparisons positively identify the product of the TES gene as an N-terminal motor kinesin, containing a tropomyosin domain. RT-PCR expression analysis reveals TES mRNA to be expressed in all parts of the Arabidopsis plant throughout the life cycle reflecting a level of redundancy also found in many kinesins. Strikingly, database searches have identified a second kinesin with TES-like features (named TES-like Kinesin 1 (TLK1)) on chromosome 1 of Arabidopsis, and a closely homologous rice genomic sequence. Phylogenetic analysis reveals TES, TLK1 and the rice sequence to form a stand-alone group, very distinct from other plant kinesins. It is envisaged that TLK genes can be used in similar ways to TES genes.

In addition, we have identified a rice homologue of the TES gene and a maize EST that forms part of a maize homologue. It is envisaged that these rice and maize homologues, and homologues in other plants, will be applicable in the same way as Arabidopsis TES.

Accordingly, the invention provides:

a plant in which, by virtue of modulation of the expression of a TETRASPORE (TES) or TES-like (TLK) gene, or of modulation of the activity of a protein encoded by such a gene, tetrad formation in the anther is disrupted, or is capable of being disrupted, such that callose cross-wall formation in the tetrad fails to the extent that fusion between two or more of the four microspore nuclei may occur.

### BRIEF DESCRIPTION OF THE DRAWINGS

FIG. 1: TETRASPORE coding region (schematic)

Stud and tes3 contain a single amino acid subsitution (C-T) and create stop codons. tes1, which has a 7 bp deletion, and tes4, which contains a 10 bp deletion, alter the reading frame and create stop codons at 38 bp and 40 bp downstream respectively.

FIG. 2: RT-PCR expression studies on TES

TP: four week young plants, R: roots, ST: stems, L: rosette leaves, YB: young buds before pollen mitosis 1, OB: buds after pollen mitosis 1, FL: open flowers. TES was found to be expressed in all tissue studies, suggesting that expression is constitutive.

FIG. 3: Protein line up of TES and homologues

Line 1 (SEQ ID NO: 1): tes protein=TES, Line 2 (SEQ ID NO: 2): Os=rice homologue, Line 3 (SEQ ID NO: 3): At=TLK1

FIG. 4: Genomic sequence of wild-type TES in Columbia-3 background and sequence of tes1 mutant

FIG. 5: Genomic sequence of WS2.R TES and TES-4 mutant

FIG. 6: Genomic sequence of LER-R TES, and TES-3 and STUD-mutants

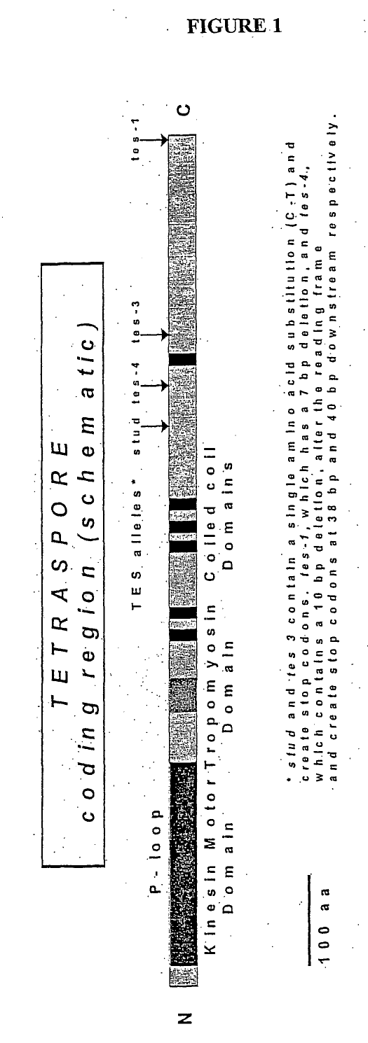

### DETAILED DESCRIPTION OF THE INVENTION

Vectors and Chimeric Genes

Genes and Polynucleotides According to the Invention

The TES gene whose genomic sequence is provided herein was cloned from Arabidopsis. However, the skilled person will appreciate that homologous genes will exist in other species. Two homologues have already been identified, namely the rice homologue of SEQ ID No. 2 and the Arabidopsis TLK1 gene. It will be appreciated that the rice homologue of SEQ ID No. 2 is a TES or TLK gene as defined herein. In particular, it is envisaged that further homologous genes will exist the plants mentioned in the section below entitled “Plants of the invention”. Based on the genomic DNA sequence and the amino acid sequence of TES provided herein, a skilled person would readily be able to design probes and primers to identify and obtain homologues in other plant species. Such homologues form part of the invention.

A preferred homologue of the invention or polynucleotide of the invention, which may be isolated form, generally:

(a) encodes a polypeptide sequence as set out in SEQ ID NO: 1, 2 or 3; or

(b) has a cDNA sequence having 60% or more homology to a sequence of (a); or

(c) has a cDNA sequence capable of hybridising selectively to the complement of a sequence of (a); and

when functional, encodes a kinesin protein; and, when non-functional, causes the plant to fail to form callose cross-walls in the tetrad.

Hybridisable Sequences

A polynucleotide of the invention may hybridise selectively to a coding sequence of (a) at a level significantly above background. Background hybridisation may occur, for example because of other cDNAs present in a cDNA library. The signal level generated is typically at least 10 fold, preferably at least 100 fold, as that generated by background hybridisation. The intensity of interaction may be measured, for example by radiolabelling the probe, e.g. with ³²P. Selective hybridisation is typically achieved using conditions of medium to high stringency (for example 0.03M sodium chloride and 0.03M sodium citrate at from about 50° C. to about 60° C., for example 45 to 50, 50 to 55 or 55 to 60° C., e.g. at 50 or 60° C.

However, such hybridisation may be carried out under any suitable conditions known in the art (see Sambrook et al, 1989, Molecular Cloning: A Laboratory Manual). For example, if high stringency is required, suitable conditions include 0.2×SSX at around 60° C., for example 40 to 50° C., 50 to 60° C. or 60 to 70° C., e.g. at 50 or 60° C. If lower stringency is required, suitable conditions include 2×SSC at around 60° C., for example 40 to 50° C., 50 to 60° C. or 60 to 70° C., e.g. at 50 or 60° C.

Stringency typically occurs in a range from about Tm-5° C. (5° C. below the melting temperature (Tm) of the two sequences hybridising to each other in a duplex) to about 20° C. to 25° C. below Tm. Thus, according to the invention, a hybridisable sequence may be one which hybridises at a temperature of from Tm to Tm-25° C., e.g. Tm to Tm-5° C., Tm-5 to Tm-10° C., Tm-10 to Tm-20° C. or Tm-20 to Tm-25° C.

Homologous Sequences

A polynucleotide sequence of the invention, will typically comprise a coding sequence at least 60% or 70%, preferably at least 80 or 90% and more preferably at least 95, 98 or 99%, homologous to a coding sequence of (a) above.

Such homology will preferably apply over a region of at least 20, preferably at least 50, for instance 100 to 500 or more, contiguous nucleotides.

Methods of measuring nucleic acid and polypeptides homology are well known in the art. These methods can be applied to measurement of homology for both polypeptides and nucleic acids of the invention. For example, the UWGCG Package provides the BESTFIT program which can be used to calculate homology (Devereux et al, 1984, Nucleic Acids Research 12, p.387-395).

Similarly, the PILEUP and BLAST algorithms can be used to line up sequences (for example as described in Altschul, S. F., 1993, J Mol. Evol. 30:290-300; Altschul, S. F. et al, 1990) J. Mol. Biol. 215:403-410).

Many different settings are possible for such programs. According to the invention, the default settings may be used.

In more detail, the BLAST algorithm is suitable for determining sequence similarity and it is described in Altschul et al (1990) J. Mol. Biol. 215:403-410. Software for performing BLAST analyses is publicly available through the National Center for Biotechnology Information (http://www.ncbi/nlm.hih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g. Karlin and Altschul (1993) Proc. Natl. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a nucleic acid is considered similar to a fused gene or cDNA if the smallest sum probability in comparison of the test nucleic acid to a fused nucleic acid is less than about 1, preferably less than about 0.1, more preferably less than about 0.01, and most preferably less than about 0.001.

Fragments

Also included within the scope of the invention are sequences which are fragments of the sequences of (a) to (c) above but have the properties of the polypeptides of invention as defined above.

Degenerate Sequences

Also included within the scope of the invention are sequences that differ from those of (b) and (c) above but which, because of the degeneracy of the genetic code, encode the same polypeptides.

Complementary Sequences

In addition, the invention provides polynucleotides having sequences complementary to any of the above-mentioned sequences. Such polynucleotides may be useful in antisense and/or RNAi applications as discussed herein.

Further Properties

Polynucleotides of the invention may comprise DNA or RNA. They may also be polynucleotides which include within them synthetic or modified nucleotides.

Polynucleotides of the invention may be used to produce a primer, e.g. a PCR primer, a primer for an alternative amplification reaction, a probe, e.g. labelled with a revealing label by conventional means using radioactive or non-radioactive labels, or the polynucleotides may be cloned into vectors. Such primers, probes and other fragments will preferably be at least 10, preferably at least 15 or 20, for example at least 25, 30 or 40 nucleotides in length. These will be useful in identifying species homologues and allelic variants as discussed above.

Polynucleotides such as a DNA polynucleotides and primers according to the invention may be produced recombinantly, synthetically, or by any means available to those of skill in the art. They may also be cloned by standard techniques. The polynucleotides are typically provided in isolated and/or purified form.

In general, primers will be produced by synthetic means, involving a stepwise manufacture of the desired nucleic acid sequence one nucleotide at a time. Techniques for accomplishing this using automated techniques are readily available in the art.

Genomic clones corresponding to the homologues of the invention and containing, for example introns and promoter regions are also aspects of the invention and may also be produced using recombinant means, for example using PCR (polymerase chain reaction) cloning techniques.

Polynucleotides which are not 100% homologous to the sequences of the present invention but fall within the scope of the invention, as described above, can be obtained in a number of ways, for example by probing cDNA or genomic libraries from other plant species with probes derived from SEQ ID NO: 1, 2, or 3. Degenerate probes can be prepared by means known in the art to take into account the possibility of degenerate variation between the DNA sequences of SEQ ID NO: 1, 2, or 3 and the sequences being probed for under conditions of medium to high stringency (for example 0.03M sodium chloride and 0.03M sodium citrate at from about 50° C. to about 60° C.), or other suitable conditions (e.g. as described above).

Allelic variants and species homologues may also be obtained using degenerate PCR which will use primers designed to target sequences within the variants and homologues encoding likely conserved amino acid sequences. Likely conserved sequences can be predicted from aligning the amino acid sequences of the invention with each other and/or with those of any homologous sequences known in the art. The primers will contain one or more degenerate positions and will be used at stringency conditions lower than those used for cloning sequences with single sequence primers against known sequences.

Alternatively, such polynucleotides may be obtained by site-directed mutagenesis. This may be useful where, for example silent codon changes are required to sequences to optimise codon preferences for a particular host cell in which the polynucleotide sequences are being expressed. Other sequences may be desired in order to introduce restriction enzyme recognition sites, or to alter the properties or function of the polypeptides encoded by the polynucleotides.

The invention further provides double stranded polynucleotides comprising a polynucleotide of the invention and its complement.

Polynucleotides, probes or primers of the invention may carry a revealing label. Suitable labels include radiosotopes such as ³²P or ³⁵S, enzyme labels, or other protein labels such as biotin. Such labels may be added to polynucleotides, probes or primers of the invention and may be detected using techniques known per se.

Polypeptides of the Invention

Polypeptides of the invention may be encoded by polypeptides as described above. A polypeptide of the invention may consist essentially of the amino acid sequence set out in SEQ ID NO: 1 or a substantially homologous sequence, or of a fragment of either of these sequences, as long as the properties of the invention are maintained.

In particular, a polypeptide of the invention may comprise:

(a) the polypeptide sequence of SEQ ID NO: 1, 2 or 3;

(b) a polypeptide sequence at least 60, 70, 80, 90, 95, 98 or 99% homologous to, a polypeptide of (a); or

(c) an allelic variant or species homologue of a sequence of (a).

Allelic Variants

An allelic variant will be a variant which occurs naturally and which will function in a substantially similar manner to the protein of SEQ ID NO: 1. Similarly, a species homologue of the protein will be the equivalent protein which occurs naturally in another species.

Homologues

A polypeptide of the invention is preferably at least 60% homologous to the protein of SEQ ID NO: 1, 2 or 3, more preferably at least 80 or 90% and more preferably still at least 95, 97 or 99% homologous thereto over a region of at least 20, preferably at least 30, for instance at least 40, 60 or 100 or more contiguous amino acids. Methods of measuring protein homology are well known in the art and it will be understood by those of skill in the art that in the present context, homology is calculated on the basis of amino acid identity (sometimes referred to as “hard homology”).

Degrees of homology can be measured by well-known methods, as discussed herein for polynucleotide sequences.

The sequence of the polypeptides of SEQ ID NOs: 1, 2 and 3 and of the allelic variants and species homologues can be modified to provide further polypeptides of the invention.

Substitutions

Amino acid substitutions may be made, for example from 1, 2 or 3 to 10, 20 or 30 substitutions. For example, a total of up to 1, 2, 5, 10 or 20 amino acids may be substituted over a length of 50, 100 or 200 amino acids in the polypeptides. For example, up to 20 amino acids substituted over any length of 50 amino acids. The modified polypeptide generally retains the neurological properties of the invention, as defined herein. Conservative substitutions may be made, for example according to the following table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Fragments

Polypeptides of the invention also include fragments of the above-mentioned fall length polypeptides and variants thereof, including fragments of the sequence set out in SEQ ID NO1: 1, 2 or 3. Such fragments typically retain the properties of the invention.

Suitable fragments will generally be at least about 20, e.g. at least 20, 50 or 100 amino acids in size. Polypeptide fragments of the polypeptides of SEQ ID NOs: 1, 2 and 3 and allelic and species variants thereof may contain one or more (e.g. 2, 3, 5, 5 to 10 or more) substitutions, deletions or insertions, including conservative substitutions. Each substitution, insertion or deletion may be of any length, e.g. 1, 2, 3, 4, 5, 5 to 10 or 10 to 20 amino acids in length.

Isolation and Purification

Polypeptides and polynucleotides of the invention may be in a substantially isolated form. It will be understood that they may be mixed with carriers or diluents which will not interfere with the intended purpose of the polypeptide and still be regarded as substantially isolated. A polypeptide or polynucleotide of the invention may also be in a substantially purified form, in which case it will generally comprise the polypeptide or polynucleotide, as the case may be, in a preparation in which more than 70%, e.g. more than 80, 90, 95, 98 or 99% of the polypeptide in the preparation is a polypeptide of the invention.

Production of Polypeptides

Polypeptides of the invention may be produced in any suitable manner. It is preferred that they be produced recombinantly from polynucleotides of the invention by expression in a suitable host cell, e.g. a bacterial, yeast or plant cell, preferably a plant cell, e.g. of a species mentioned herein, either in culture or in planta. Polypeptides may be recovered and, optionally, purified by techniques known in the art. This can be done using known techniques.

Replicable Vectors

Polynucleotides of the invention can be incorporated into recombinant replicable vectors. Such vectors may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and cultivating the host cell under conditions which bring about replication of the vector. The vector may be recovered from the host cell. Suitable host cells are described below in connection with expression vectors. Bacterial cells, especially E. coli are preferred.

Chimeric Genes and Expression Vectors

In particular, the invention provides a chimeric gene comprising, operably linked to one or more regulatory sequences capable of securing its expression in a cell, a coding sequence encoding a polypeptide of the invention.

Such chimeric genes can, in turn, be incorporated into expression vectors. Thus, preferably, a polynucleotide of the invention is operably linked to regulatory sequences capable of effecting the expression of the coding sequence by a host cell. Such chimeric genes and expression vectors can be used to express the polypeptides of the invention.

The term “operably linked” refers to a juxtaposition wherein the components described are in a relationship permitting them to function in their intended manner. A regulatory sequence “operably linked” to a coding sequence is positioned in such a way that expression of the coding sequence is achieved under conditions compatible with the regulatory sequences.

Such chimeric genes and expression vectors may be introduced into a suitable host cell to provide for expression of a polypeptide or polypeptide fragment of the invention, as described below.

The vectors may be for example, plasmid, cosmid, virus or phage vectors provided with an origin of replication, preferably a promoter for the expression of the said polynucleotide and optionally an enhancer and/or a regulator of the promoter. A terminator sequence may also be present, as may a polyadenylation sequence. The vectors may contain one or more selectable marker genes, for example antibiotic ampicillin resistance genes. These will generally be operably linked to regulatory sequences capable of securing their expression in the host cell, as described herein for the coding sequences of the invention.

Vectors may be used in vitro, for example for the production of RNA or used to transfect or transform a host cell. The vector may also be adapted to be used in vivo, for example for generation of transgenic plants of the invention.

So far as plasmid vectors are concerned, plasmids derived from the Ti plasmid of Agrobacterium tumefaciens are especially preferred, as are plasmids derived from the Ri plasmid of Agrobacterium rhizogens.

A further embodiment of the invention provides host cells transformed or transfected with the vectors for the replication and expression of polynucleotides of the invention. The cells will be chosen to be compatible with the said vector and may for example be prokaryotic (bacterial), plant,. yeast, insect or mammalian cells, bacterial and plant cells being preferred.

Polynucleotides according to the invention may also be inserted into the vectors described above in an antisense orientation in order to provide for the production of antisense RNA. Antisense RNA or other antisense polynucleotides may also be produced by synthetic means. Such antisense polynucleotides may be used in a method of reducing the levels of expression of polypeptides having the sequence of SEQ ID NO: 1, or variants or species homologues thereof in planta.

An antisense polynucleotide of the invention may be capable of hybridising to mRNA of a gene of the invention, or a variant or species homologue thereof, as defined herein (a “target” mRNA) and may thus inhibit expression by interfering with one or more aspects of mRNA metabolism including transcription, mRNA processing, mRNA transport from the nucleus, translation or mRNA degradation.

The antisense polynucleotide may be DNA, but is typically RNA. The antisense polynucleotide may be provided as single or double stranded polynucleotide. The antisense polynucleotide typically hybridises to the target mRNA to form a duplex (typically an RNA-RNA duplex) which can cause direct inhibition of translation and/or destabilisation of the mRNA. Such a duplex may be susceptible to degradation by nucleases.

The antisense polynucleotide may hybridise to all or part of the target mRNA. Typically the antisense polynucleotide hybridises to the ribosome binding region or the coding region of the target mRNA. The polynucleotide may be complementary to all of or a region of the target mRNA. For example, the polynucleotide may be the exact complement of all or a part of target mRNA. However, absolute complementary is not required and polynucleotides which have sufficient complementarity to form a duplex having a melting temperature of greater than 20° C., 30° C., or 40° C. under physiological conditions are particularly suitable for use in the present invention. The polynucleotide may be a polynucleotide which hybridises to the target mRNA under conditions of medium to high stringency such as 0.03M sodium chloride and 0.03M sodium citrate at from about 50° C. to about 60° C.

In one preferred embodiment the antisense polynucleotide sequence is complementary to the entire coding sequence of the target mRNA and to the nucleotides of the mRNA immediately 5′ of the coding sequence. However, the polynucleotide may hybridise to all or part of the 5′- or 3′-untranslated region of the mRNA. The antisense polynucleotide may be of any length but will typically be from 6 to 40 nucleotides in length. More preferably it will be from 12 to 20 nucleotides in length. The polynucleotide may be at least 40, for example at least 60 or at least 80, nucleotides in length and up to 100, 200, 300, 400, 500, 1000 or more nucleotides in length. In one embodiment the length of the antisense oligonucleotide is the same as that of the target mRNA or up to a few nucleotides, such as 5 or 10 nucleotides, shorter than the mRNA.

Promoters and other regulatory elements may be selected to be compatible with the host cell for which the expression vector is designed.

Promoters suitable for use in plant cells may be derived, for example, from plants or from bacteria that associate with plants or from plant viruses. Thus, promoters from Agrobacterium spp. including the nopaline synthase (nos), octopine synthase (ocs) and mannopine synthase (mas) promoters are preferred. Also preferred are plant promoters such as the ribulose bisphosphate small subunit promoter (rubisco ssu), histone promoters (EP-A-0 507,698), the rice actin promoter (U.S. Pat. No. 5,641,876) and the phaseolin promoter. Also preferred are plant viral promoters such as the cauliflower mosaic virus (CAMV) 35S and 19S promoters, and the circovirus promoter (AU-A-689,311).

Depending on the pattern of expression desired, promoters may be constitutive, tissue- or stage-specific; and/or inducible. For example, strong constitutive expression in plants can be obtained with the CAMV 35S, Rubisco ssu, or histone promoters mentioned above. Also, tissue-specific or stage-specific promoters may be used to target expression of polypeptides of the invention to particular tissues in a transgenic plant or to particular stages in its development. Promoters specific to the anther, or to early anther development are particularly advantageous.

Inducible promoters are particularly preferred. Alcohol-inducible and herbicide-inducible promoters are available. Chemically inducible promoters such as those activated by herbicide safeners may also be used, for example the maize GST 27 promoter (WO97/11189), the maize In2-1 promoter (WO90/11361), the maize In2-2 promoter (De Veylder et al, Plant Cell Physiology, Vol.38, pp568-577 (1997).

Especially where expression in plant cells is desired, other regulatory signals may also be incorporated in the vector, for example a terminator and/or polyadenylation site. Preferred terminators include the nos terminator and the histone terminator of EP-A-0 633,317 although other terminators functional in plant cells may also be used. Additionally, sequences encoding secretory signals or transit peptides may be included. On expression, these elements direct secretion from the cell or target the polypeptide of the invention to a particular location within the cell. For example, sequences may be added to target the expressed polypeptide to the nucleus or plastids (e.g. chloroplasts) of a plant cell.

Some examples are signal-peptide encoding DNA/RNA sequences which target proteins to the extracellular matrix of the plant cell, such as the signal sequence of the Nicotiana plumbaginifolia extension gene; signal peptides which target proteins to the vacuole, like those of the sweet potato sporamin gene and the barley lectin gene; signal peptides which cause proteins to be secreted such as that of PRIb; or the barley α-amylase leader sequence; and signal peptides which target proteins to the plastids such as that of rapeseed enoyl-Acp reductase.

Typically, therefore, a chimeric gene comprises the following elements in 5′ to 3′ orientation: a promoter functional in a host (preferably plant) cell, as defined above, a polynucleotide of the invention and a terminator functional in said cell, as defined above.

Other elements, for example enhancers, may also be present in a vector of the invention. Enhancers include the tobacco etch virus (TEV) enhancer and the tobacco. mosaic virus (TMV) enhancer (WO87/07644).

Similarly, an origin of replication may be present. Sequences capable of securing integration into a cells genome, e.g. Agrobacterium tumefaciens T-DNA sequences may be present.

Further, selectable marker genes, under control of their own regulatory sequences may be included. These include antibiotic resistance genes. Examples include genes that confer resistance to the antibiotics kanamycin and/or neomycin (e.g. the nptI and nptII genes) or chloramphenicol (e.g. the CAT gene). Herbicide resistance genes may also be used as selectable markers. Notably, genes conferring resistance to herbicides such as bialaphos, glyphosate or an isoxazole herbicide may be used. Particular examples are described in EP-A-0 242,236, EP-A-0 242,246, GB-A-2,197,653, WO91/02701, WO95/06128, WO96/38567 and WO97/04103. Likewise, scorable marker genes may be present. Some examples are the β-glucuronidase (GUS) β-galactosidase luciferase and green fluorescent protein (gfp) genes.

Expression in Host Cells

Expression in the host cell may be transient although, preferably, integration of the polynucleotide or chimeric gene of the invention into the cell's genome is achieved.

Cell culture will take place under standard conditions. Commercially available cultural media for cell culture are widely available and can be used in accordance with manufacturers' instructions.

Plants of the Invention

The present invention is in principle applicable to any plant species, notably arable crop species, tree species and species used in horticulture, especially ornamentals. Preferred dicotyledonous crop plants include tomato; potato; sugarbeet cassava; cruciferous crops, including oilseed rape; linseed; tobacco; sunflower; fibre crops such as cotton; and leguminous crops such as peas, beans, especially soybean, and alfalfa. Brassicas are particularly preferred. Preferred monocotyledonous plants include graminaceous plants such as wheat, maize, rice, oats, barley, rye, sorghum, triticale and sugar cane. Maize is particularly preferred.

The invention is also particularly useful in the context of transgenic trees, where male sterility is of benefit because it prevents dispersal of transgenic pollen.

Transformation/Regeneration Techniques

The cell used for transformation may be from any suitable organism, preferably a plant as defined herein and may be in any form. For example, it may be an isolated cell, e.g. a protoplast, or it may be part of a plant tissue, e.g. a callus, for example a solid or liquid callus culture, or a tissue excised from a plant, or it may be part of a whole plant. It may, for example, be part of an embryo, or a meristem, e.g. an apical meristem of a shoot. Transformation may thus give rise to a chimeric tissue or plant in which some cells are transgenic and some are not.

Transformation Techniques

Cell transformation may be achieved by any suitable transformation method, for example the transformation techniques described herein. Preferred transformation techniques include electroporation of plant protoplasts (Taylor and Walbot, 1985), PEG-based procedures (Golds et al, 1993), microinjection (Neuhas et al, 1987; Potrykus et al, 1985), injection by galinstan expansion femtosyringe (Knoblauch et al, 1999), Agrobacterium-mediated transformation and particle bombardment. Particle bombardment is particularly preferred.

Selection of Transformed Cells

Cells generated by the transformation techniques discussed above will typically be present in chimeric tissues, and thus will be surrounded by other non-transformed cells. Standard selection techniques using co-transforming selectable and/or scorible markers can then be used to identify and obtain transformed cells.

Regeneration and Breeding

Transformed cells may be regenerated into a transgenic plant by techniques known in the art. These may involve the use of plant growth substances such as auxins, giberellins and/or cytokinins to stimulate the growth and/or division of the transgenic cell. Similarly, techniques such as somatic embryogenesis and meristem culture may be used. Regeneration techniques are well known in the art and examples can be found in, e.g. U.S. Pat. No. 4,459,355, U.S. Pat. No. 4,536,475, U.S. Pat. No. 5,464,763, U.S. Pat No. 5, 177,010, U.S. Pat. No. 5,187,073, EP 267,159, EP 604, 662, EP 672, 752, U.S. Pat. No. 4,945,050, U.S. Pat No. 5,036,006, U.S. Pat. No. 5,100,792, U.S. Pat. No. 5,371,014, U.S. Pat No. 5,478,744, U.S. Pat. No 5,179,022, U.S. Pat. No. 5,565,346, U.S. Pat. No. 5,484,956, U.S. Pat. No. 5,508,468, U.S. Pat. No. 5,538,877, U.S. Pat. No. 5,554,798, U.S. Pat. No. 5,489,520, U.S. Pat. No. 5,510,318, U.S. Pat. No. 5,204,253, U.S. Pat. No. 5,405,765, EP 442,174, EP 486,233, EP 486,234, EP 539,563, EP 674,725, WO91/02071, WO 95/06128 and WO 95/32977.

In many such techniques, one step is the formation of a callus, i.e. a plant tissue comprising expanding and/or dividing cells. Such calli are a further aspect of the invention as are other types of plant cell cultures and plant parts. Thus, for example, the invention provides transgenic plant tissues and parts, including embryos, meristems, seeds, shoots, roots, stems, leaves and flower parts. These may be chimeric in the sense that some of their cells are transgenic and some are not.

Regeneration procedures will typically involve the selection of transgenic cells by means of marker genes. The regeneration step gives rise to a first generation transgenic plant. The invention also provides methods of obtaining transgenic plants of further generations from this first generation plant. These are known as progeny plants. Progeny plants of second, third, fourth, fifth, sixth and further generations may be obtained from the first generation progeny plant by any means known in the art.

Thus, the invention provides a method of obtaining a transgenic plant of the invention comprising obtaining a second-generation transgenic progeny plant from a first-generation progeny plant of the invention, and optionally obtaining transgenic plants of one or more further generations from the second-generation progeny plant thus obtained.

Such progeny plants are desirable because the first generation plant may not have all the characteristics required for cultivation. For example, for the production of first generation transgenic plants, a plant of a taxon that is easy to transform and regenerate may be chosen. It may therefore be necessary to introduce further characteristics in one or more subsequent generations of progeny plants before a plant more suitable for cultivation is produced.

Progeny plants may be produced from their predecessors of earlier generations by any known technique. In particular, progeny plants may be produced by:

obtaining a transgenic seed from a transgenic plant of the invention belonging to a previous generation, then obtaining a transgenic progeny plant of the invention belonging to a new generation by growing up the seed; and/or

propagating clonally a transgenic plant of the invention belonging to a previous generation to give a transgenic progeny plant of the invention belonging to a new generation; and/or

crossing a first-generation transgenic plant of the invention belonging to a previous generation with another compatible plant to give a transgenic progeny plant of the invention belonging to a new generation; and optionally

obtaining transgenic progeny plants of one or more further generations from the progeny plant thus obtained.

These techniques may be used in any combination. For example, clonal propagation and sexual propagation may be used at different points in a process that gives rise to a plant suitable for cultivation. In particular, repetitive back-crossing with a plant taxon with agronomically desirable characteristics may be undertaken. Further steps of removing cells from a plant and regenerating new plants therefrom may also be carried out.

Also, further desirable characteristics may be introduced by transforming the cells, plant tissues, plants, or seeds, at any suitable stage in the above process, to introduce desirable coding sequences other than the polynucleotides of the invention. This may be carried out by conventional breeding techniques, e.g. fertilizing a plant of the invention with pollen from a plant with the desired additional characteristic. Alternatively, the characteristic can be added by further transformation of the plant obtained by the method of the invention. Preferably, different transgenes are linked to different selectable of scorable markers to allow selection for both the presence of further transgenes. Selection, regeneration and breeding techniques for nuclear transformed plants are known in the art.

Uses of Plants of the Invention

The invention also provides methods of obtaining crop products by harvesting, and optionally processing further, cells, calli, plants or seeds of the invention. By crop product is meant any useful product obtainable from a crop plant.

Such a product may be obtainable directly by harvesting or indirectly, by harvesting and further processing. Directly obtainable products include: grains, e.g. grains of monocotyledonous species, preferably graminaceous species, for example wheat, oats, rye, rice, maize, sorghum, triticale, especially wheat; other seeds; shoots, especially tubers, such as potato tubers; fruit; and other plant parts, for example as defined herein. Alternatively, such a product may be obtainable indirectly, by harvesting and further processing. Examples of products obtainable by further processing are: flour; oil; rubber; beverages such as juices and fermented and/or distilled alcoholic beverages; food products made from directly obtained or further processed material, e.g. bread made from flour or margarine made from oil; tobacco and tobacco products such as cigarettes and cigars; fibres, e.g. cotton, linen, flax and hemp fibres and textile items made therefrom; paper or timber derived from woody plants.

Additional Features of Plants of the Invention

In addition to the transgenes of the invention, plants of the invention may be transgenic in other respects. For example, they may be transformed such that they comprise genes for herbicide, insecticide or disease resistance. Preferred herbicide resistance genes may be responsible for, for example, tolerance to: Glyphosate (e.g. using an EPSP synthase gene (e.g. EP-A-0 293,358) or a glyphosate oxidoreductase (WO 92/000377) gene); or tolerance to fosametin; a dihalobenzonitrile; glufosinate, e.g. using a phosphinothrycin acetyl transferase (PAT) or glutamine synthase gene (cf. EP-A-0 242,236); asulam, e.g. using a dihydropteroate synthase gene (EP-A-0 369,367); or a sulphonylurea, e.g. using an ALS gene); diphenyl ethers such as acifluorfen or oxyfluorfen, e.g. using a protoporphyrogen oxidase gene); an oxadiazole such as oxadiazon; a cyclic imide such as chlorophthalim; a phenyl pyrazole such as TNP, or a phenopylate or carbamate analogue thereof; spectinomycin e.g. using the aadA gene, as exemplified below.

Insect resistance may be introduced, for example using genes encoding Bacillus thuringiensis (Bt) toxins. Likewise, genes for disease resistance may be introduced, e.g. as in WO91/02701 or WO95/06128.

Transformation may also lead to the introduction of a selectable marker gene i.e. marker genes that allow transformed cells to survive in the presence of agents that kill non-transformed cells. Any selectable marker gene may be used in the transforming polynucleotide of the invention. Some examples have already been given above. Typically, herbicide resistance genes, e.g. as defined above, may be used as selectable markers. Alternatively, coding regions that encode products which provide resistance to aminoglycoside antibiotics may be used as selectable marker, for example, encoded products that provide resistance to kanomycin, neomycin or chloramphenicol. The encoded polypeptide may cause morphological alterations to cultured transformed cells, such as isopentyltransferase (Kunkel et al, 1999). The encoded polypeptide may be a scorable marker, which allows transformed cells to be distinguished from non-transformed cells, generally by alteration of the transformed cell's optical properties. Any scorable marker may be used. Preferred scorable markers include, polypeptides which are able to alter the appearance or optical properties of transformed cells, for example: β-glucoronidase (i.e. the uidA:GUS gene); fluorescent proteins such as green fluorescent protein (GFP), yellow fluorescent protein (YFP) or cyan fluorescent protein (CFP); or luminescent proteins such as luciferase or aequorin. Cells with scorable optical differences can be sorted using techniques such as fluorescence activated cell sorting (FACS). In a preferred embodiment, the polynucleotide of the invention comprises a selectable marker and a scorable marker, for example, the FLARE-S marker genes which comprise aadA and GFP (Khan and Maliga, 1999).

Similarly, plants of the invention may be transformed such that they express polypeptides whose mass production is desirable, e.g. components of antibodies, or pharmaceutically active polypeptides such as interferon-gamma.

Strategies for Exploitation of TES and TLK Genes

Desirably, down-regulation of TES and/or TLK genes according to the invention will be inducible, eg by means of the techniques described herein. Constitutive down-regulation is less desirable as it will lead to the production of partially fertile plants. The key stage for down-regulation of TES and/or TLK is at the point of pollen production and fertilisation.

Another preferred technique is therefore use of recombinase-encoding constructs to excise, for example, antisense or RNAi constructs according to the invention. Recombinase systems to do this are available in the art. In outline, a construct, for example, an antisense construct of the invention, is flanked by sequences that are recognised by the recombinase enzyme. The recombinase enzyme is then capable of splicing the flanking sequences together, thus excising the construct of the invention. In a particularly preferred embodiment, down-regulation of TES and/or TLK achieved by means of an introduced antisense or RNAi construct. Of course, this plant is generally the male parent plant in any cross. The female parent plant in the cross is transformed with a construct encoding a recombinase enzyme, and the antisense or RNAi construct in the male parent plant is flanked by the sequences recognised by the recombinase. Thus, when the two plants are crossed, the recombinase is expressed in the progeny and splices out the antisense or RNAi construct. The use of recombinases in this manner is particularly advantageous because it eliminates the transgenic construct of the invention, which is beneficial from the point of view of public perception. Even more desirably, the recombinase construct may be self-excising, in that it is itself flanked by sequences which the enzyme it encodes can excise. Alternatively, the transgene can be segregated out by conventional techniques.

TES and TLK genes also have implications in increasing seed size. This is particularly desirable in graminaceous crops where it is the seed that is the part of the plant that is consumed by humans and animals. Increased seed size is desirable because it may increase overall yield and, even if it does not, it may increase seed size at the expense of the number of seeds. This is advantageous because it reduces the amount of processing required. It is envisaged that overexpression of TES and/or TLK genes will increase seed size.

Antibodies

The invention also provides monoclonal or polyclonal antibodies which specifically recognise polypeptides of the invention, and methods of making such antibodies. Antibodies of the invention bind specifically to the polypeptides of the invention.

Monoclonal antibodies may be prepared by conventional hybridoma technology using polypeptides of the invention as immunogens. Polyclonal antibodies may also be prepared by conventional means which comprise inoculating a host animal, for example a rat or a rabbit, with a polypeptide of the invention, or a fragment thereof comprising an epitope, and recovering immune serum. In order that such antibodies may be made, polypeptides may be haptenised to another polypeptide for use as immunogens in animals or humans. For the purposes of this invention, the term “antibody” includes antibody fragments such as Fv, F(ab) and F(ab)₂ fragments, as well as single-chain antibodies.

Antibodies to the polypeptides of the invention can be produced by use of the following methods. An antibody to the substance may be produced by raising antibody in a host animal against the whole substance or an antigenic epitope thereof (hereinafter “the immunogen”). Methods of producing monoclonal and polyclonal antibodies are well-known.

A method for producing a polyclonal antibody comprises immunising a suitable host animal, for example an experimental animal, with the immunogen and isolating immunoglobulins from the serum. The animal may therefore be inoculated with the immunogen, blood subsequently removed from the animal and the IgG faction purified.

A method for producing a monoclonal antibody comprises immortalising cells which produce the desired antibody. Hybridoma cells may be produced by fusing spleen cells from an inoculated experimental animal with tumour cells (Kohler and Milstein, Nature (1975) 256, 495-497).

An immortalised cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

Thus, the invention provides the use of a polypeptide of the invention, or a fragment thereof comprising an epitope, in the production of antibodies that specifically recognise a polypeptide of the invention. For these purposes, it should be noted that the fragments may not function as plant-protective polypeptides, because an epitope may be contained within a region too small to retain function as a plant-protective polypeptide. Similarly, the invention provides methods of producing antibodies by inoculating animals with a polypeptide of the invention or a fragment thereof containing an epitope and recovering immune serum. This will generate polyclonal antibodies.

Antibodies may also be generated using β-cells in vitro instead of in vivo.

Antibodies to polypeptides of the invention may also be identified by phage display techniques.

Antibodies of the invention can be used to identify compounds whose structural properties (e.g. shape, charge) correspond to those of the polypeptides of the invention. Thus, they may be used to screen for compounds that mimic the functional properties of the polypeptides of the invention.

The invention is illustrated below by means of the following Examples.

### EXAMPLES

1. Screening Recombinants of tes Individuals in F2 Mapping Population

The tes1 mutant (Columbia background) (Spielman et al (1997), supra) was crossed with wild-type Ler. F1 plants were selfed and phenotypically tes individuals in F2 were scored and genoinic DNA from each individual was extracted.

A total of 891 phenotypically tes individuals were obtained. Molecular markers ATPOX/T20P21 (CAPs), north of tes, and NIT1 (SSLP), 1cM south of tes were used to screen the mapping population. With marker ATPOX, 13 out of 891 phenotypically tes individuals were recombinants, 51 recombinants with NIT1. Primers were generated according to sequenced BAC ends within the region between ATPOX and NIT1 and used to score the recombinants from both sides and ‘walk’ towards the tes locus.

With primers from one end of BAC clone F7K15, 5 recombinants from marker ATPOX side were detected. BAC clone T5C2 has 10 kb sequence overlap with the other end of F7K15. One recombinant was observed by using primers from T5C2 end on NIT1 side. No recombinants were detected with primers from the region between the two overlapped clones.

2. Detection of Polymorphisms Between tes Alleles and Wild Type Plants (Ecotypes)

TES has been mapped to two overlapped Arabidopsis BAC clones (F7K15 and T5C2) which have been sequenced and annotated. The size of the F7K15 clone is 110800 bp containing 15 predicted proteins and 4 transposons. T5C2 is 103098 bp having 13 predicted proteins and 7 transposons.

(1) Southern blot analysis has been done on three ecotypes (Col, Ler and WS2) and corresponding tes alleles using the two BAC clones as probes to detect RFLPs associated with mutant alleles. No polymorphisms were observed with 9-20 restriction enzyme digested DNA blots.

(2) PCR based analysis (CAPs) of tes alleles and wild-type plants was conducted using primers corresponding to the sequences of F7K15 and T5C2 clones. The size of the PCR products were from 5 kb to 10 kb and they were overlapped from 0.5 kb to 1 kb. PCR products digested with restriction enzymes were screened for polymorphisms. Polymorphisms were observed within a predicted kinesin-like protein region (F7K15-60 from 27071 to 31660 bp on F7K15 BAC sequence) with restriction enzymes Alu1, Dde1 and Sau3al on alleles tes1 and tes4 (Spielman et al (1997), supra). The experimental detection of polymorphisms has been repeated and confirmed by several independent experiments.

tes1 was mutated on ecotype Col-3, while sequence data obtained from ecotype Col-0. Col-3 and Col-0 showed the same restriction digestion pattern on the tes region. This confirmed that tes1 mutations are not due to ecotype background.

3. Sequence Analysis of tes Region

The size of the tes genomic sequence is 4589 bp encoding 932 amino acids. Both strands of ˜7.5 kb region from 4 alleles and 3 ecotypes have been sequenced. Results showed that tes4 (T-DNA mutagenesis) has a 10 bp deletion, which alters the reading frame. A stop code is created at 40 bp downstream of the deletion; tes3 and stud (EMS mutagenesis) have one base pair substitutions (C-T) and place stop codons (CAA-TAA; CGA-TGA) in the kinesin motor domain. tes1 (fast neutron mutagenesis) contains 7 bp deletion causing a Frameshift and creating a stop code 38 bp downstream. Mutations of tes4, tes3 and stud are located in the third last exon of 14 exons, the tes1 mutation is in the last exon of the gene.

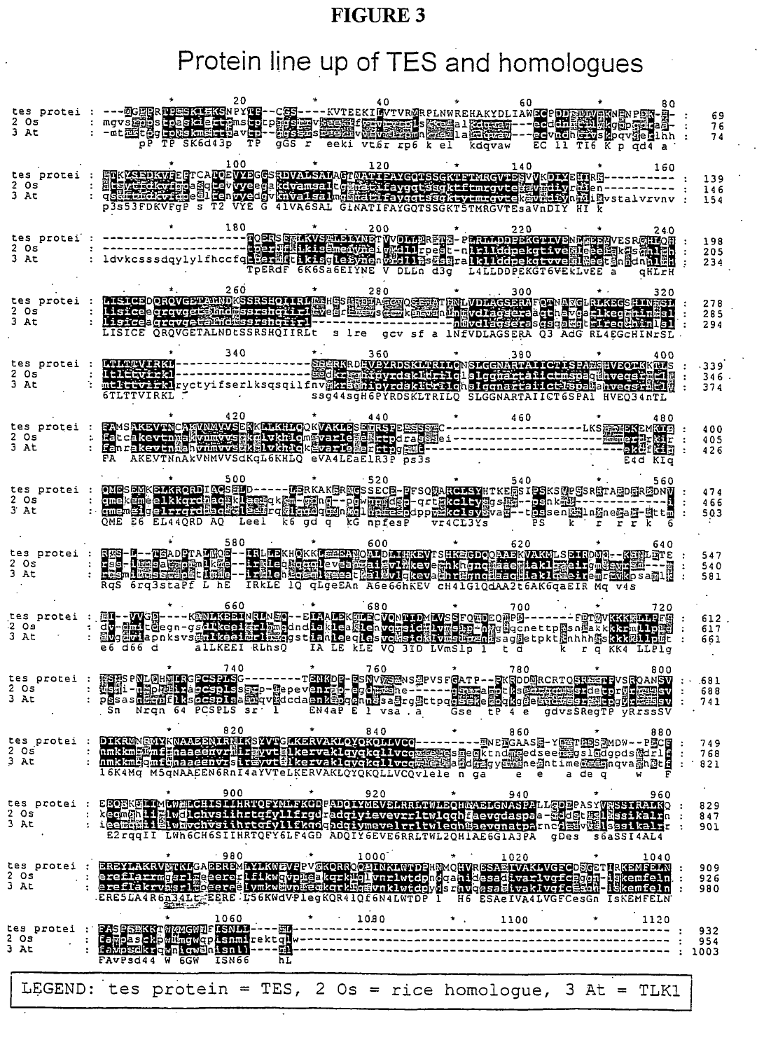

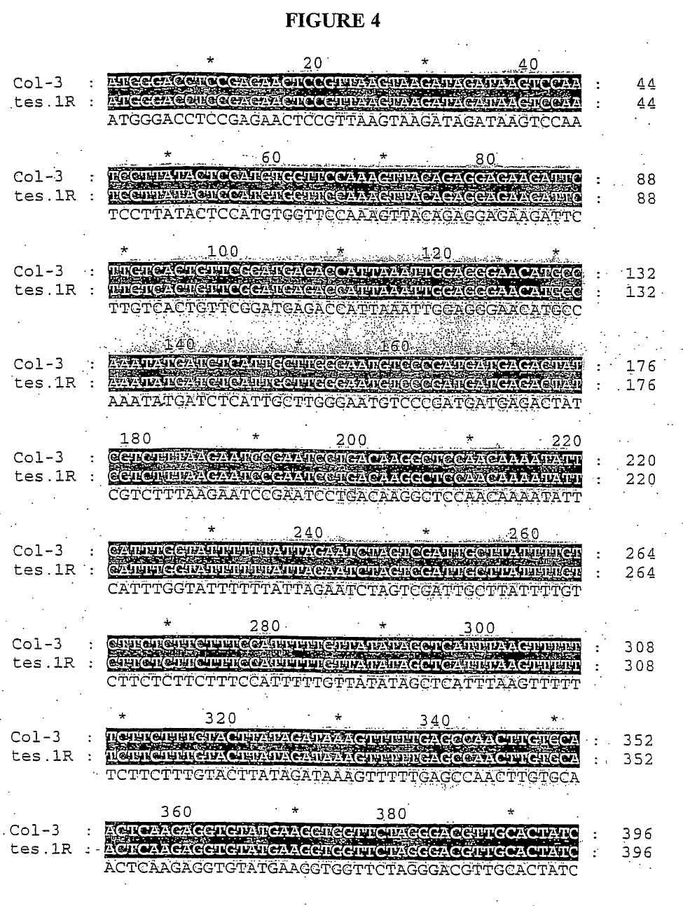

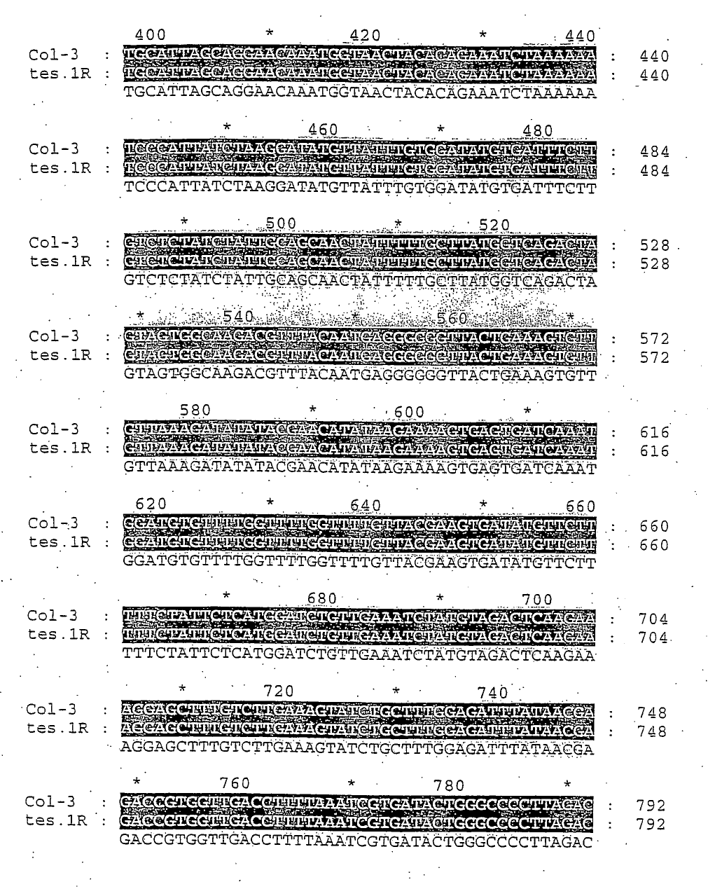

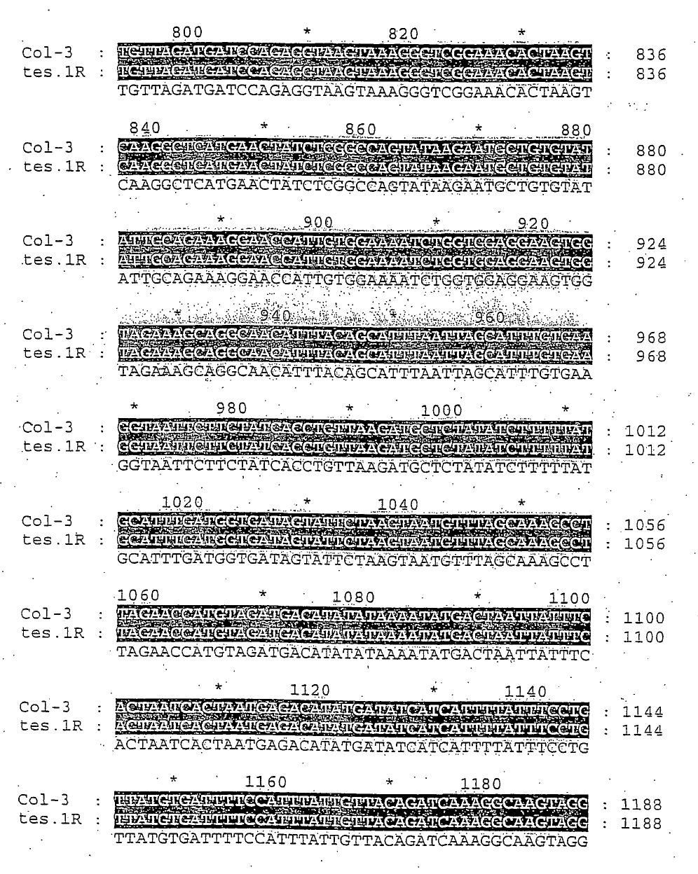

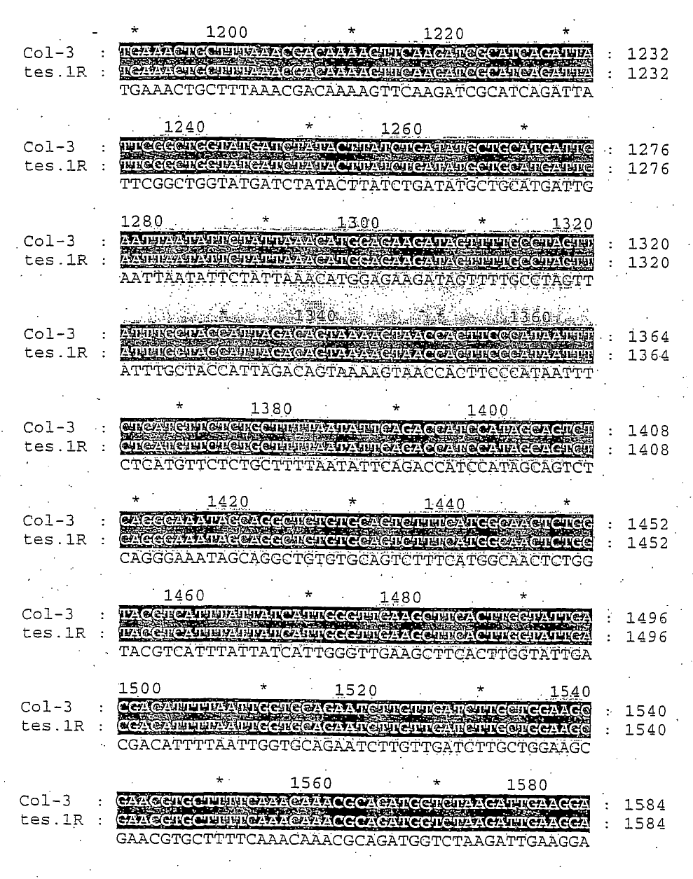

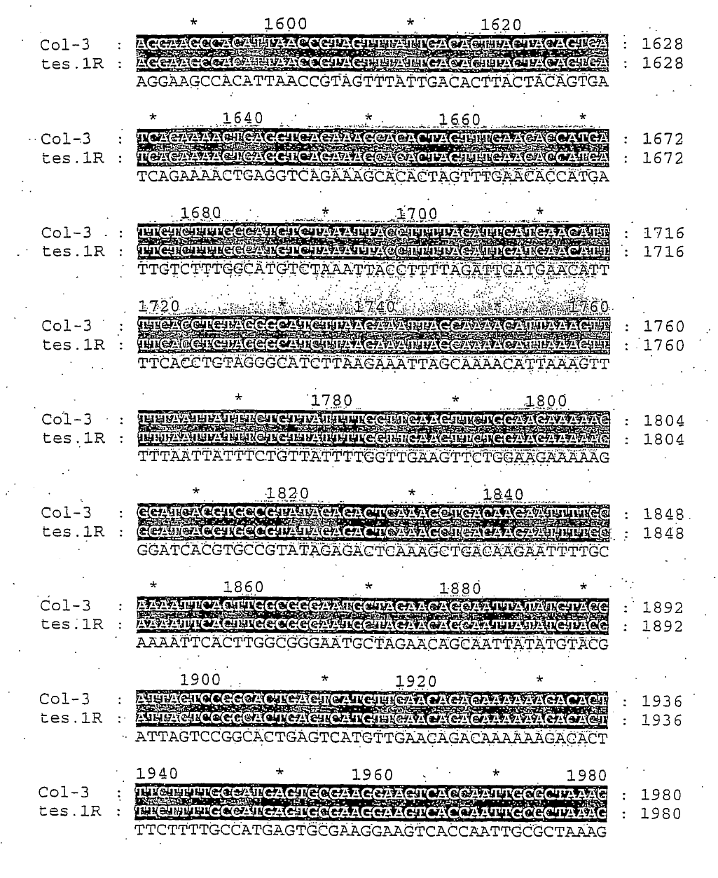

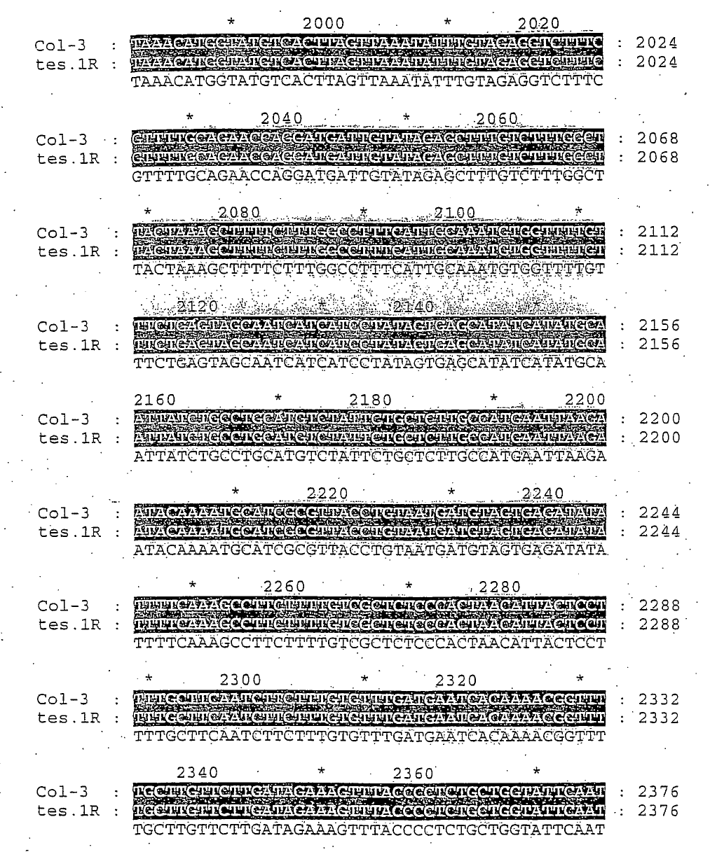

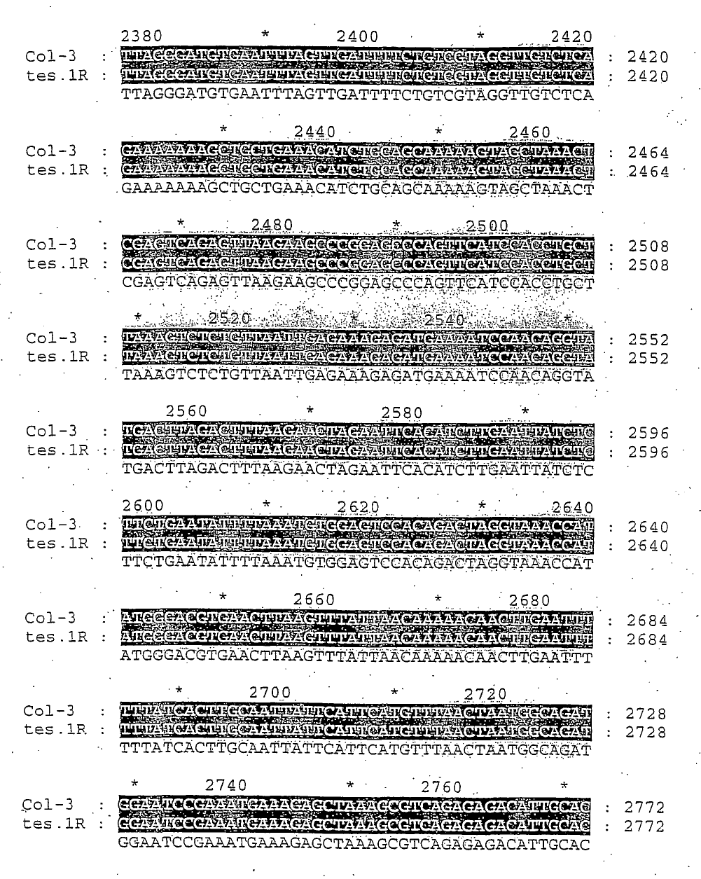

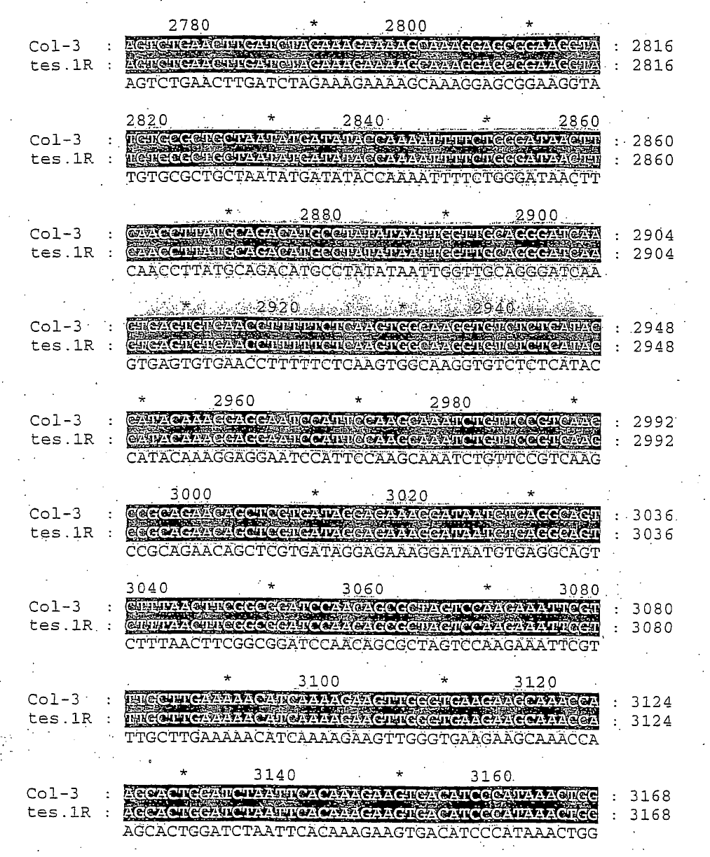

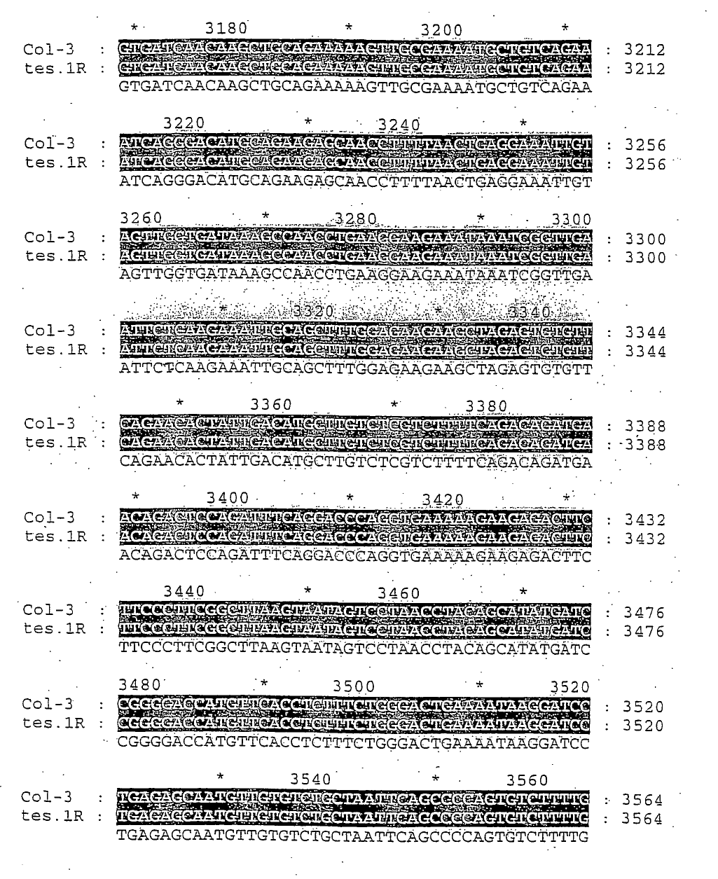

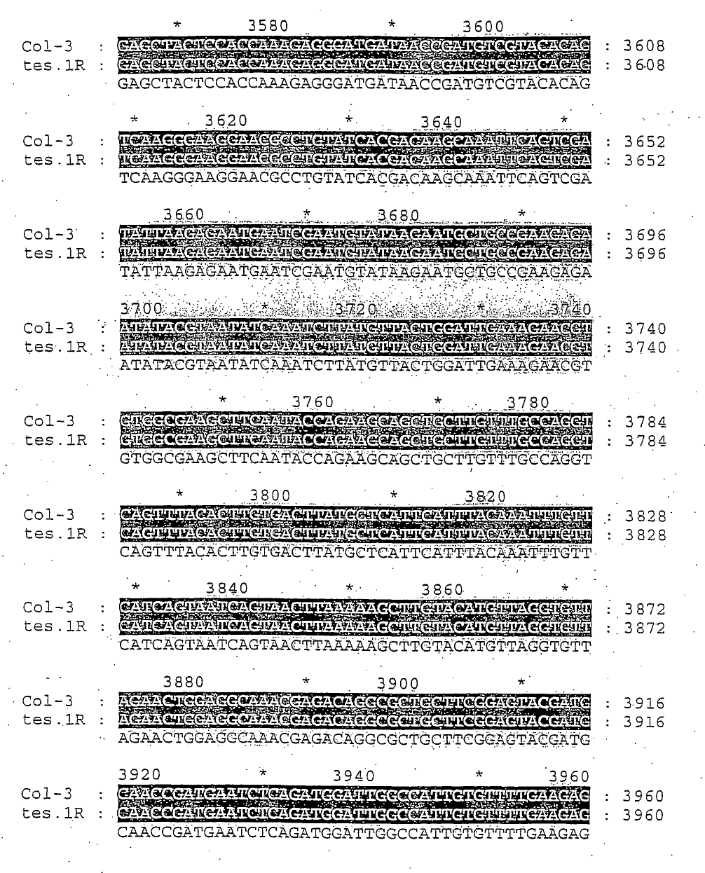

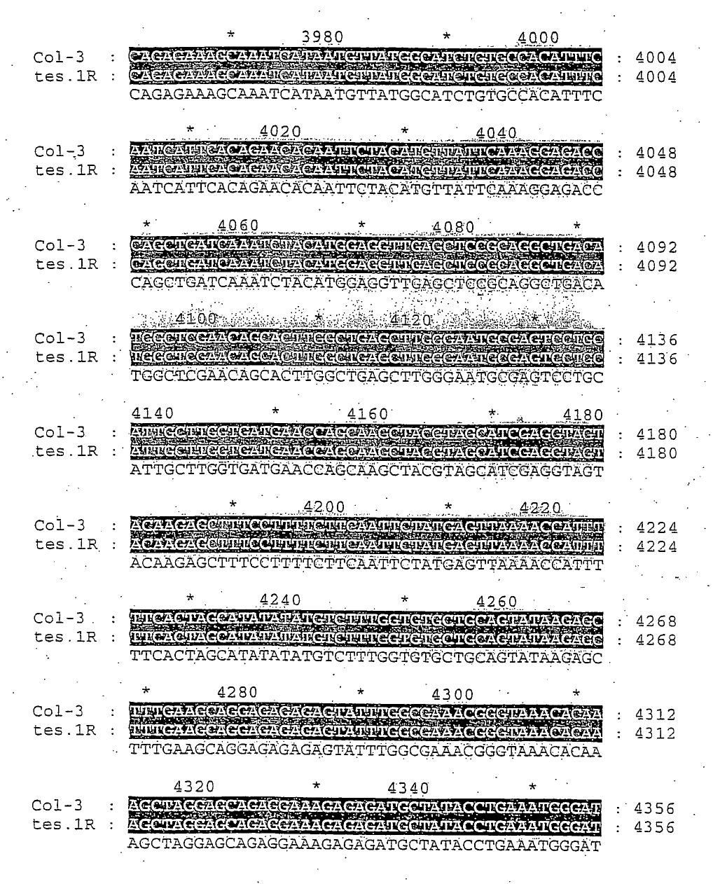

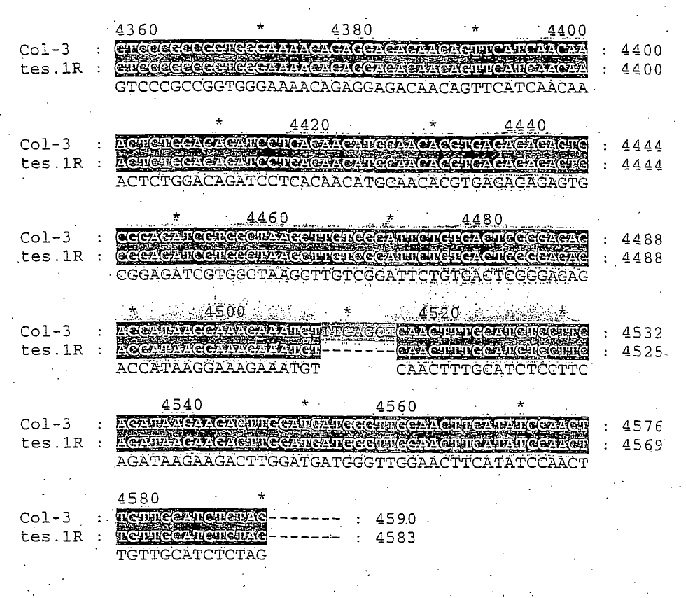

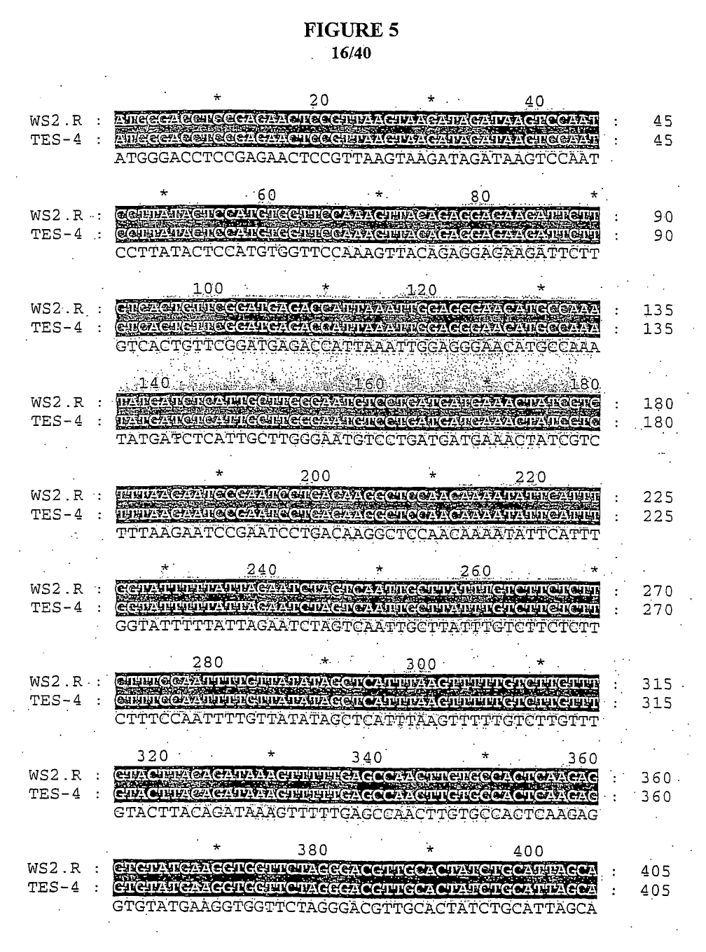

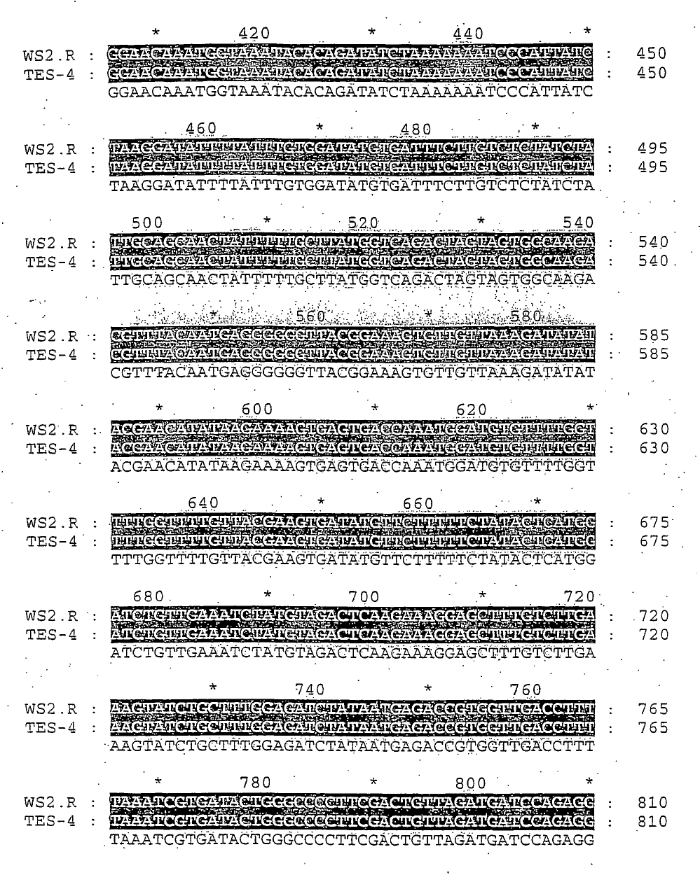

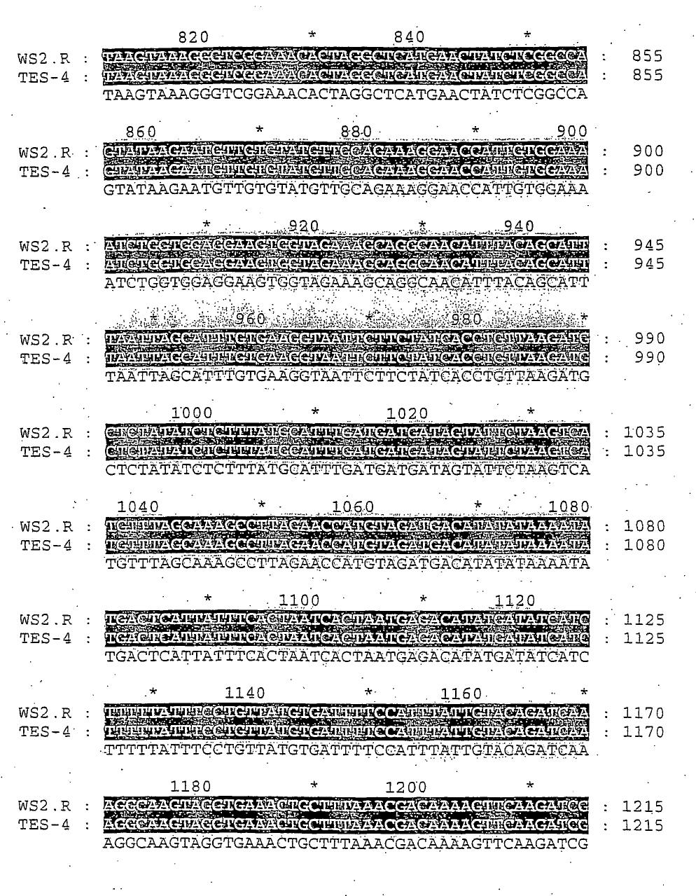

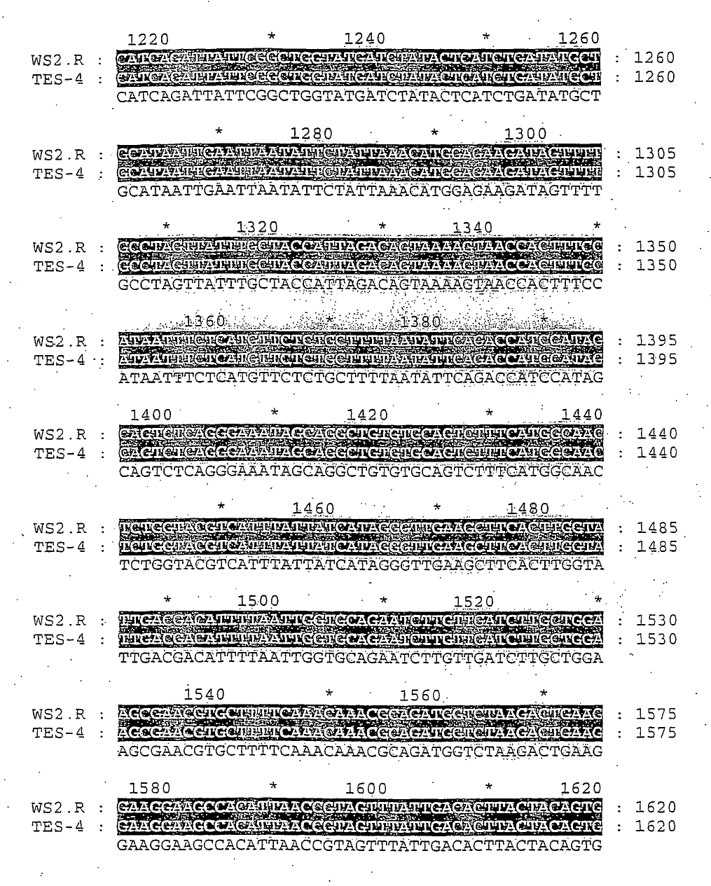

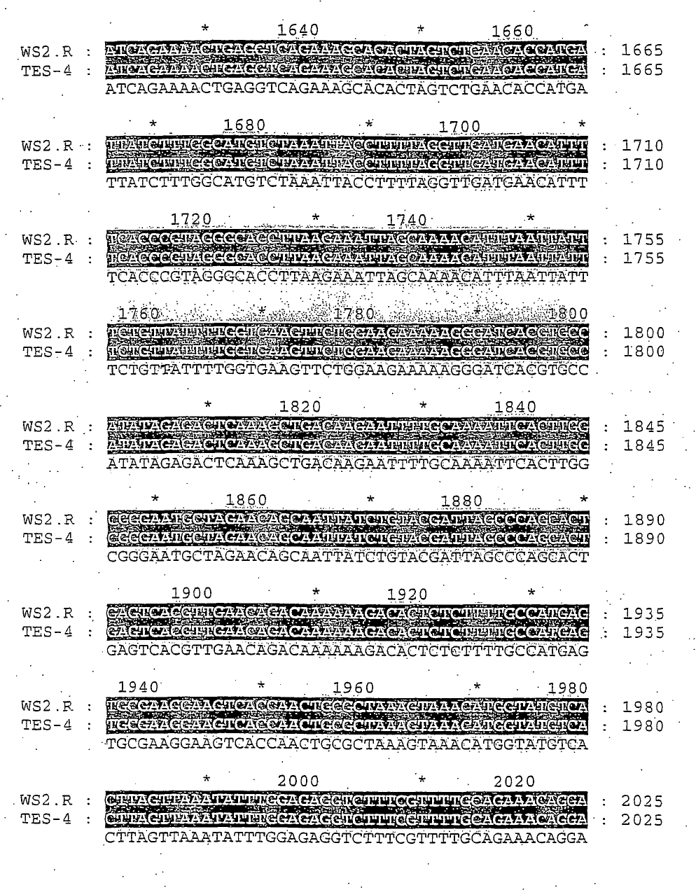

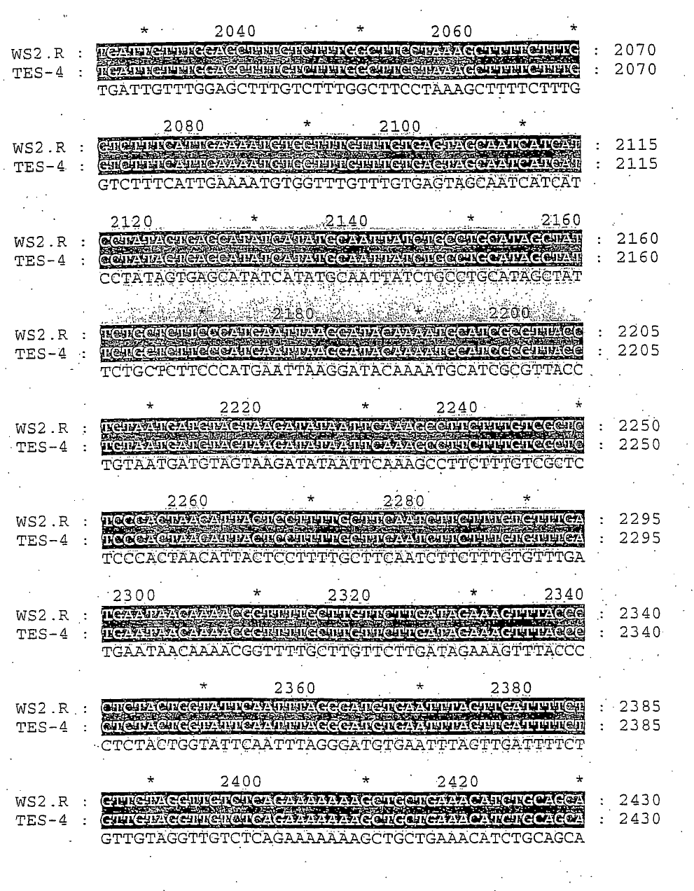

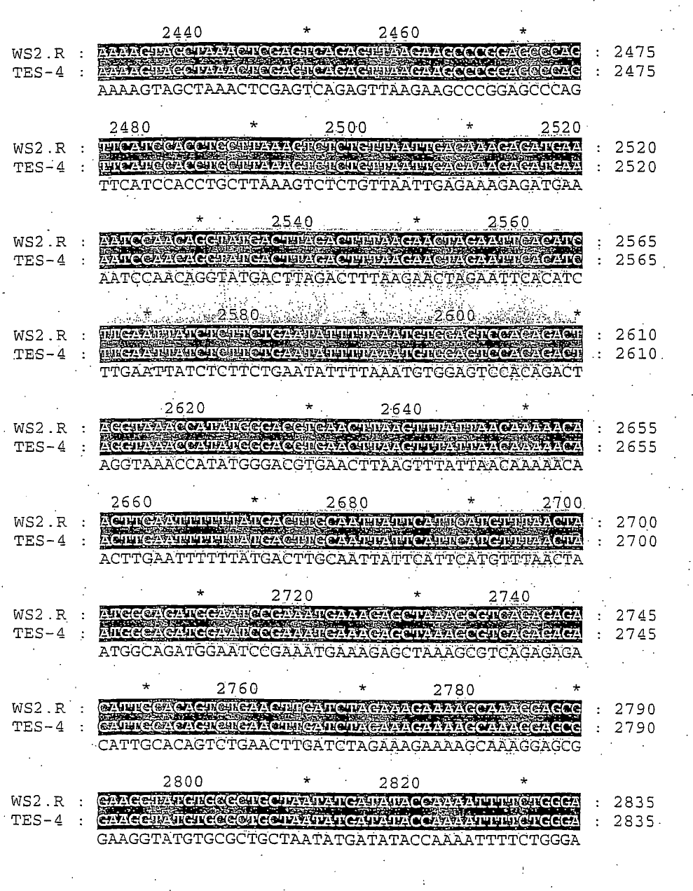

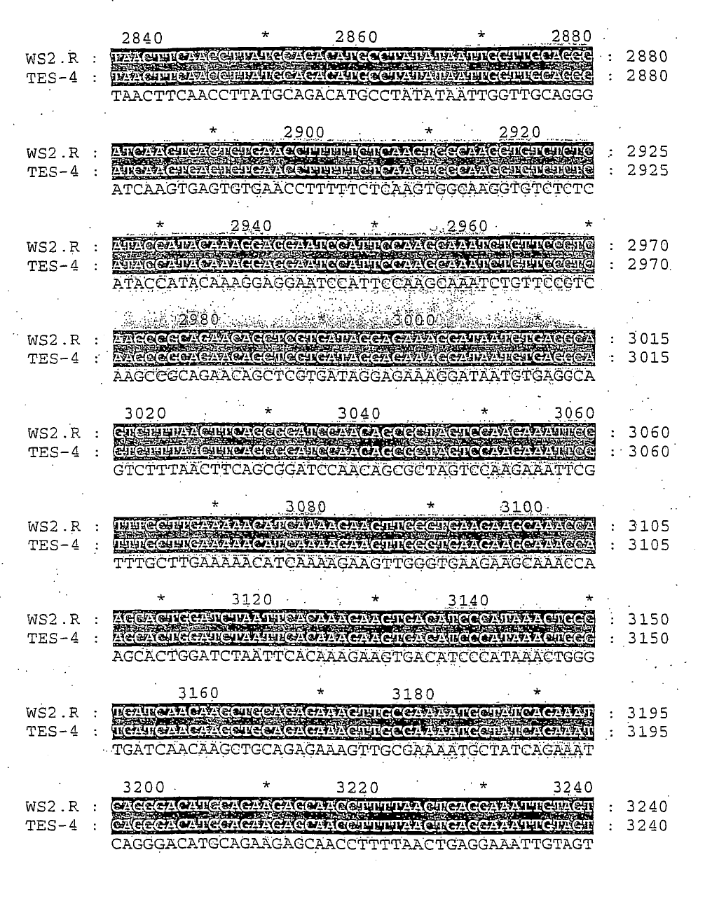

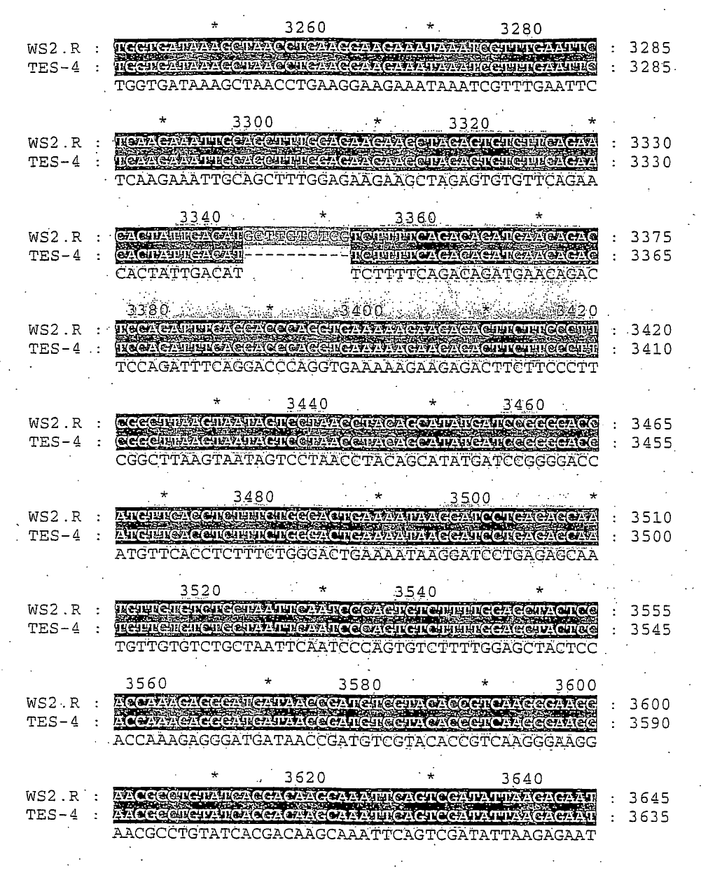

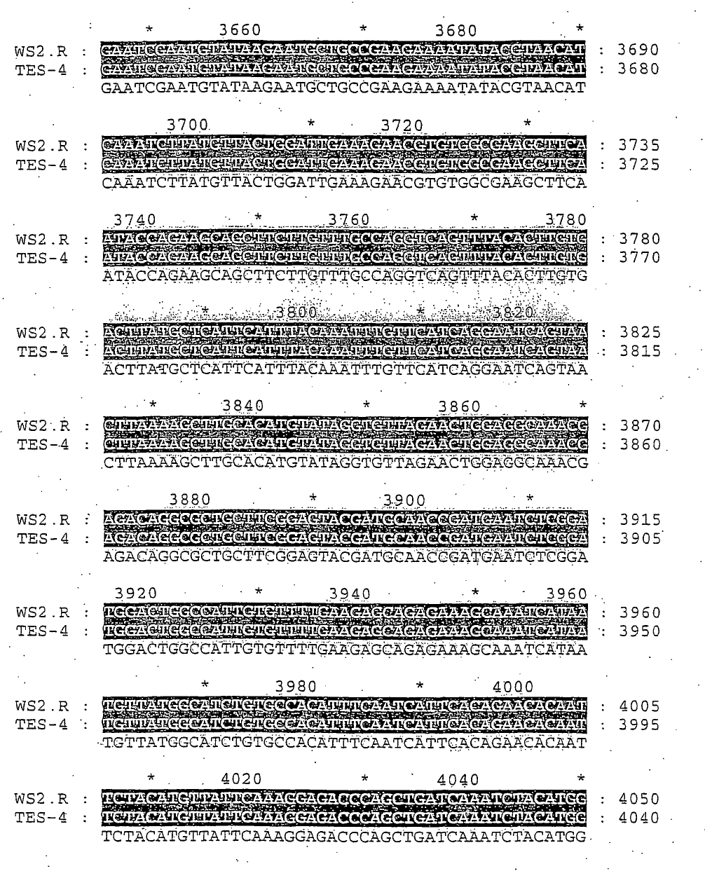

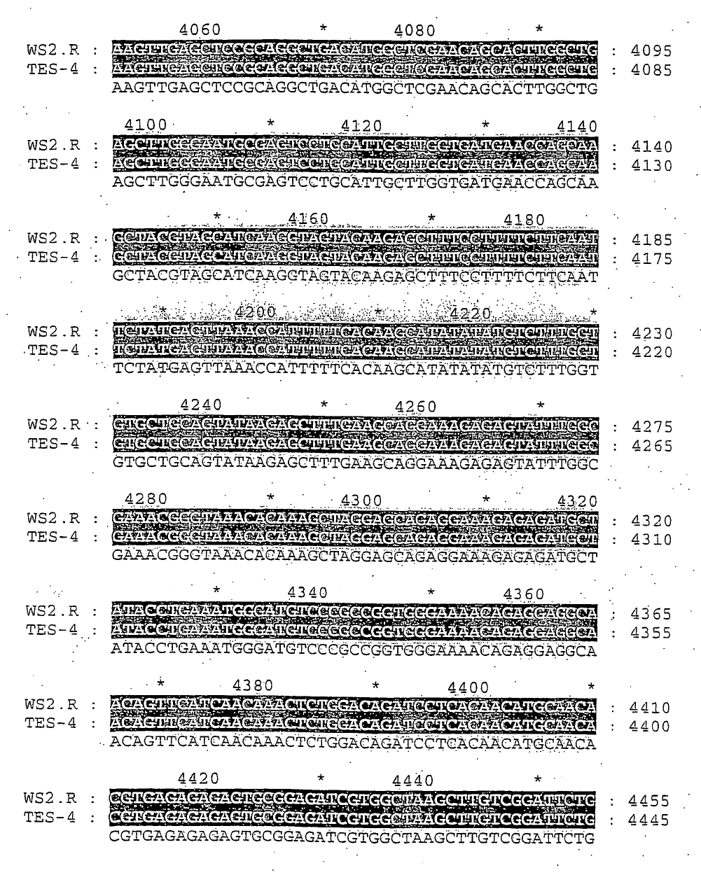

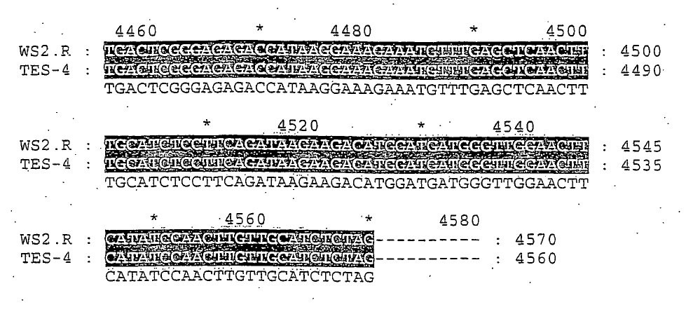

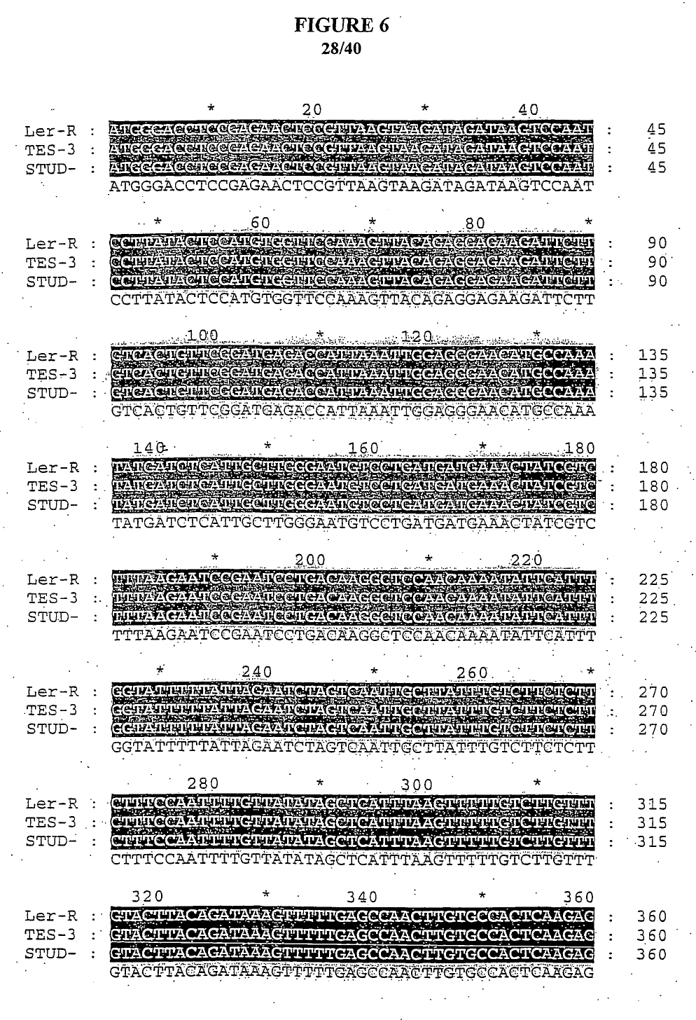

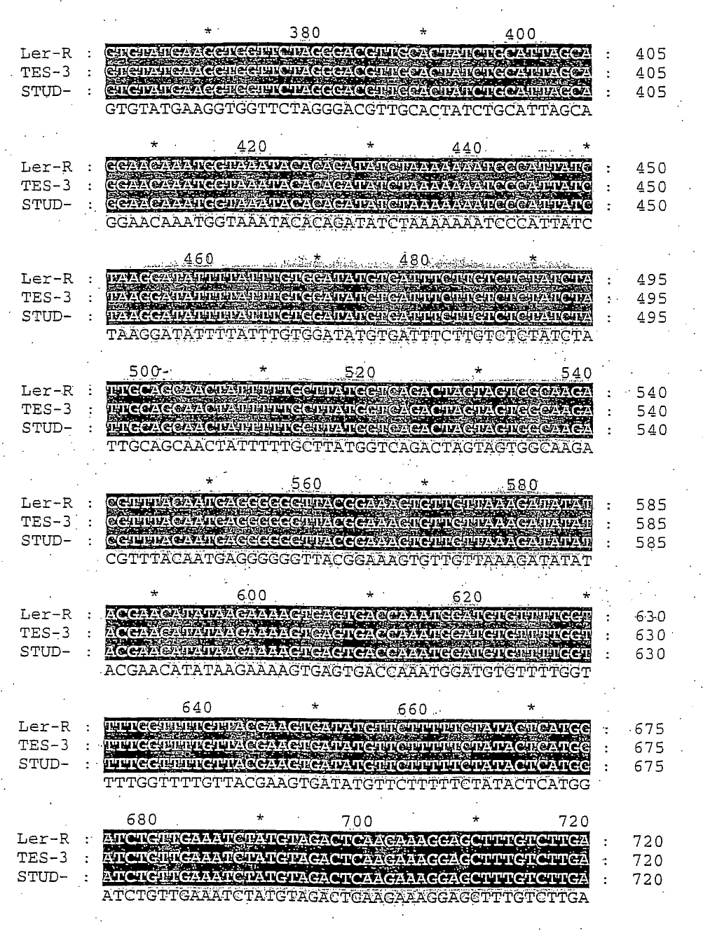

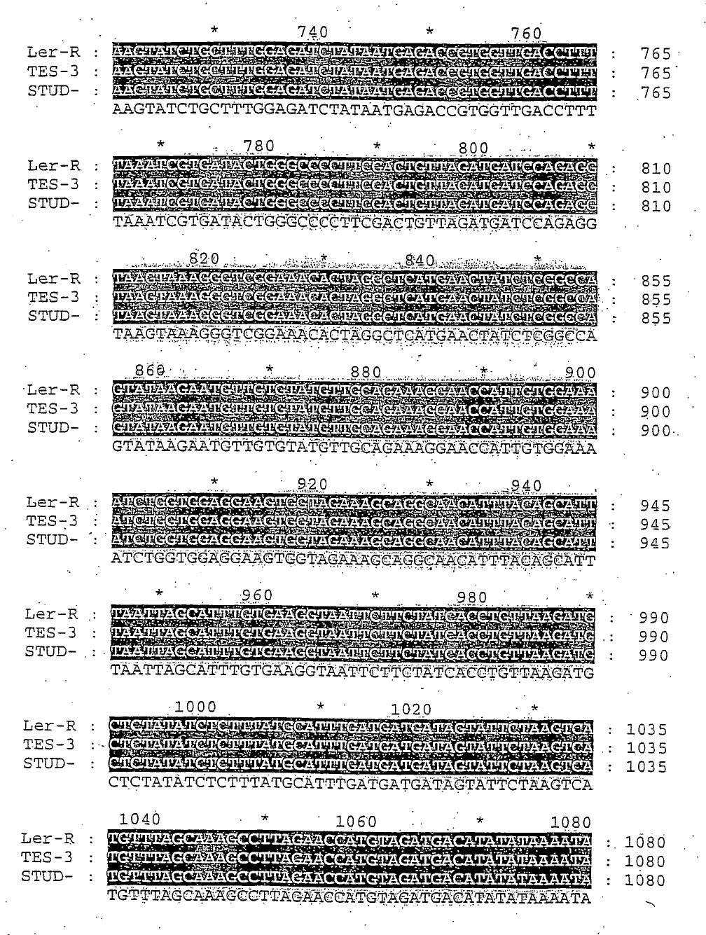

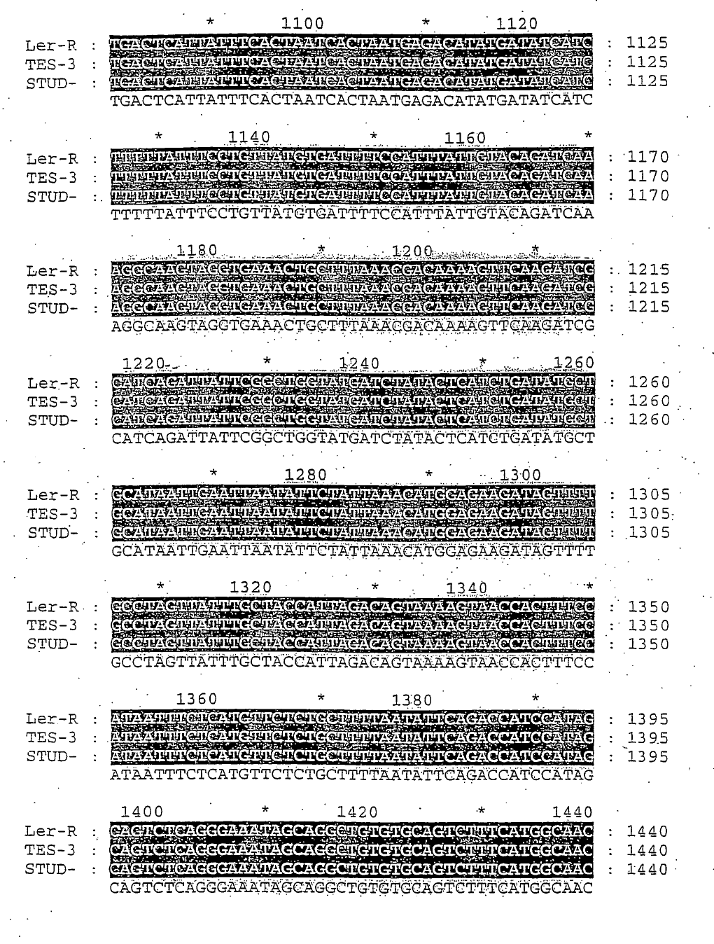

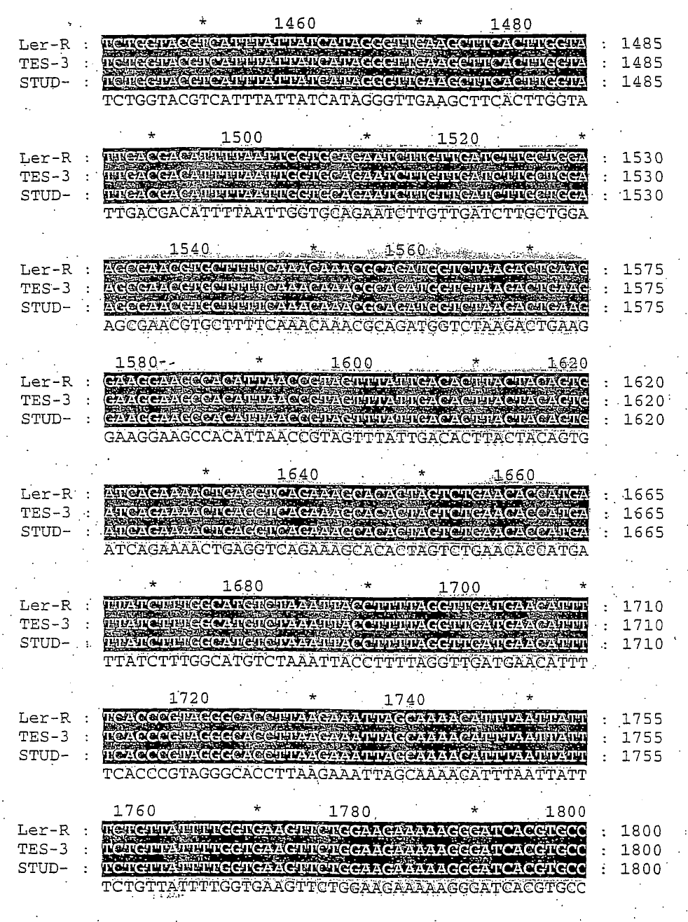

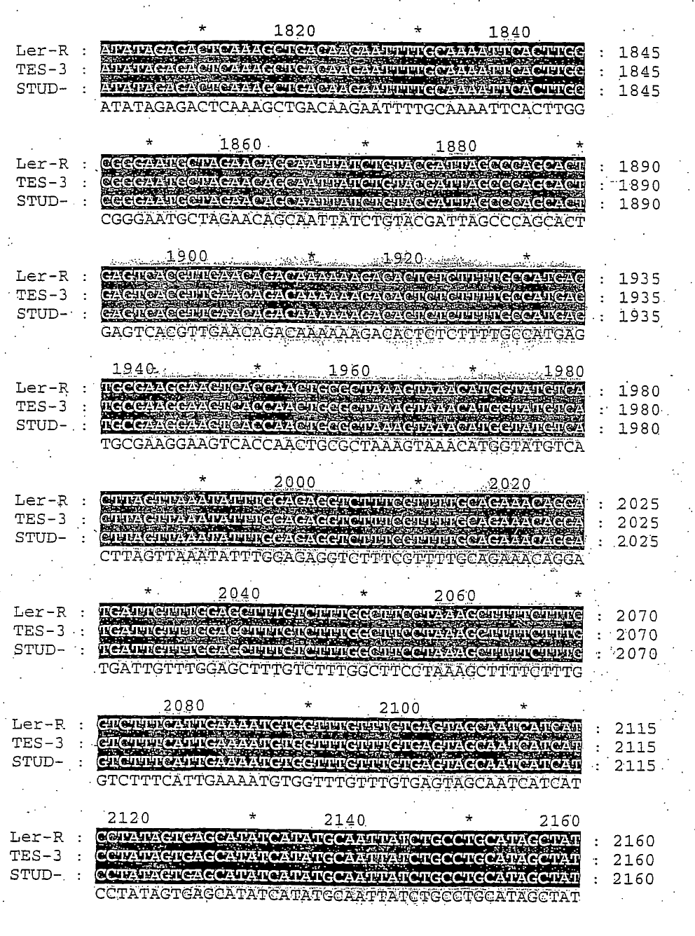

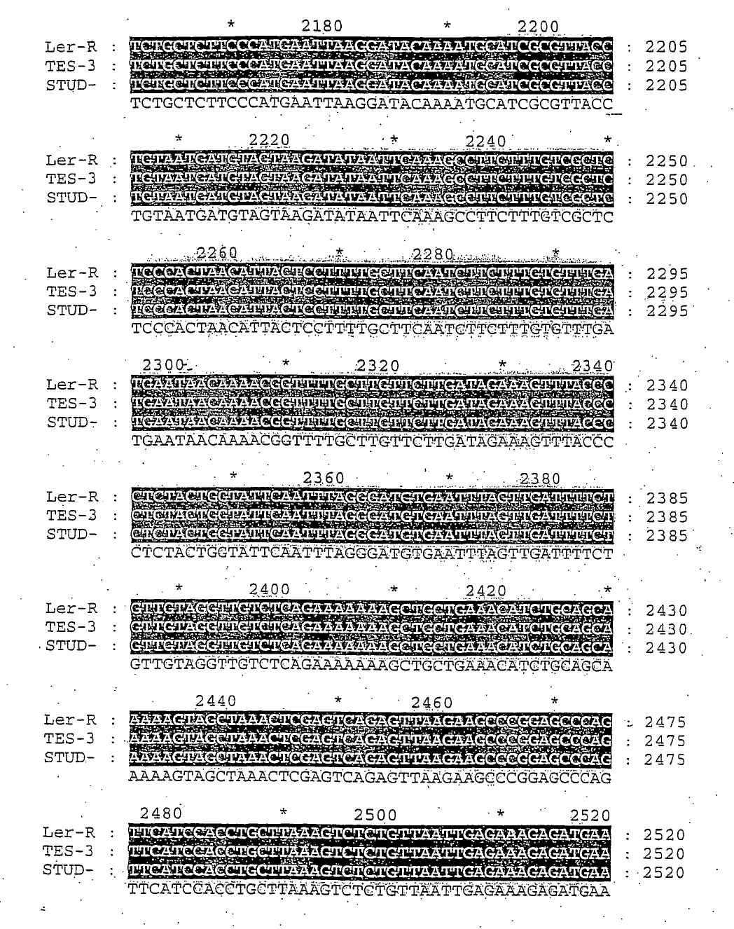

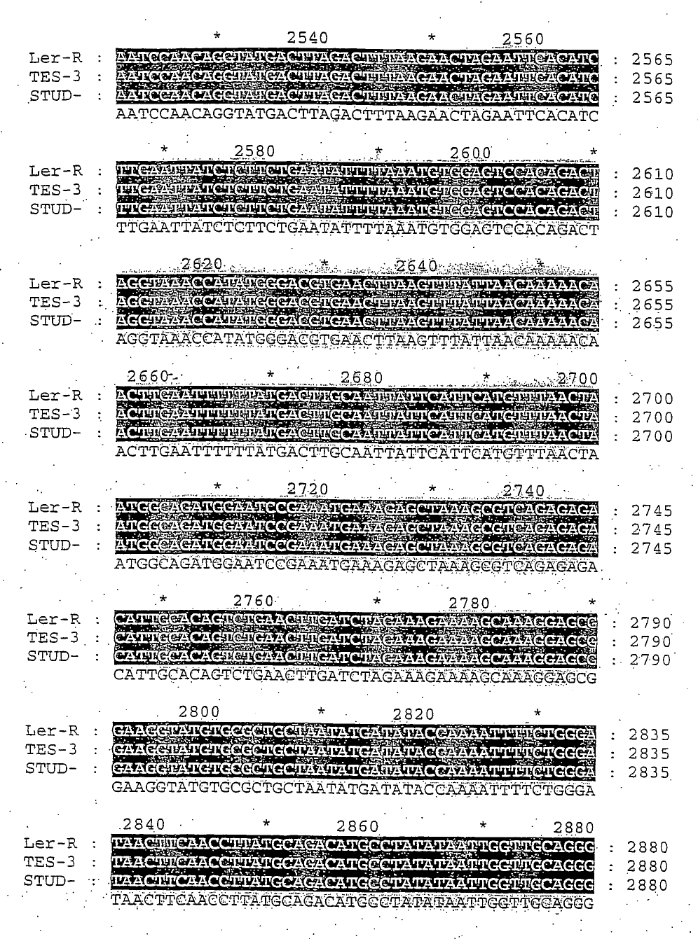

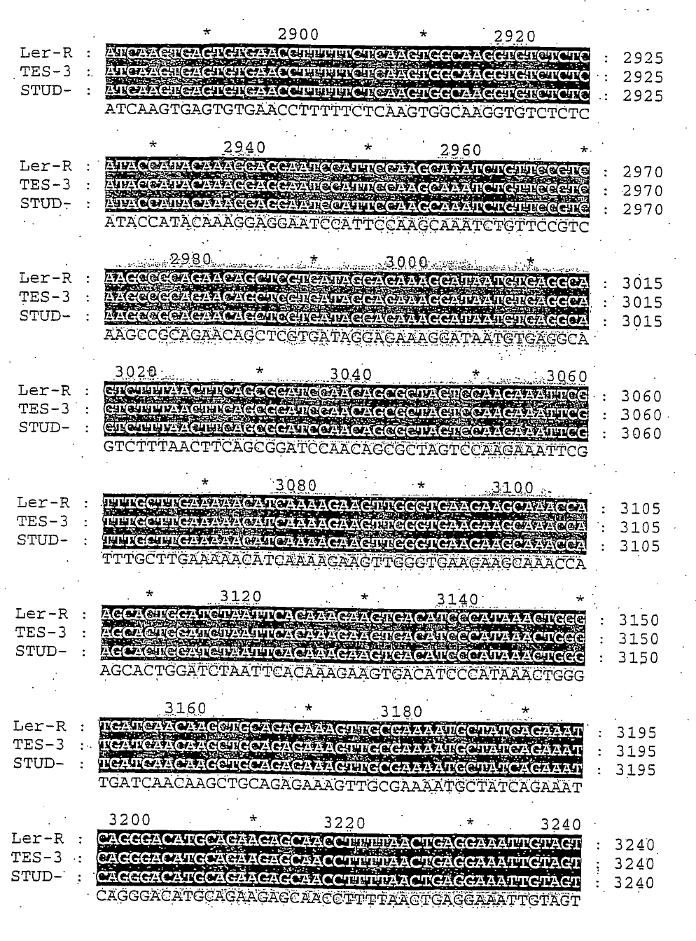

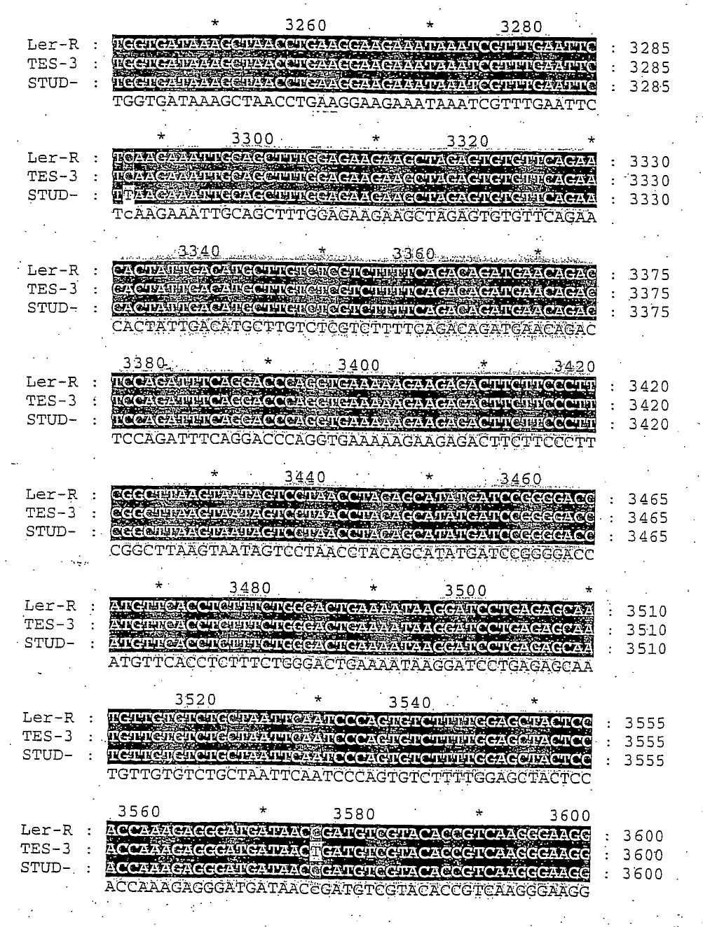

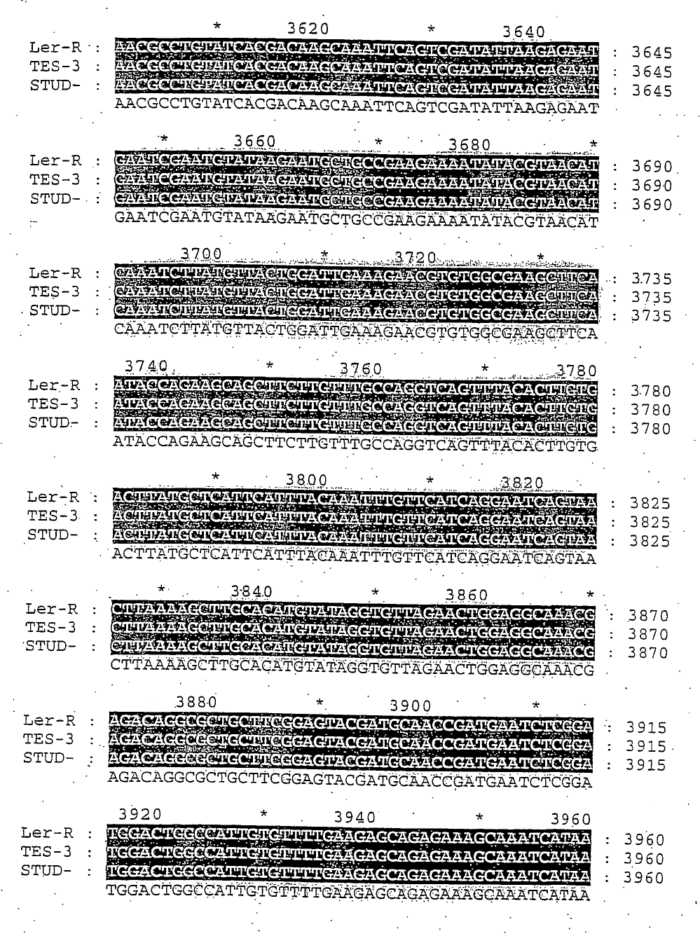

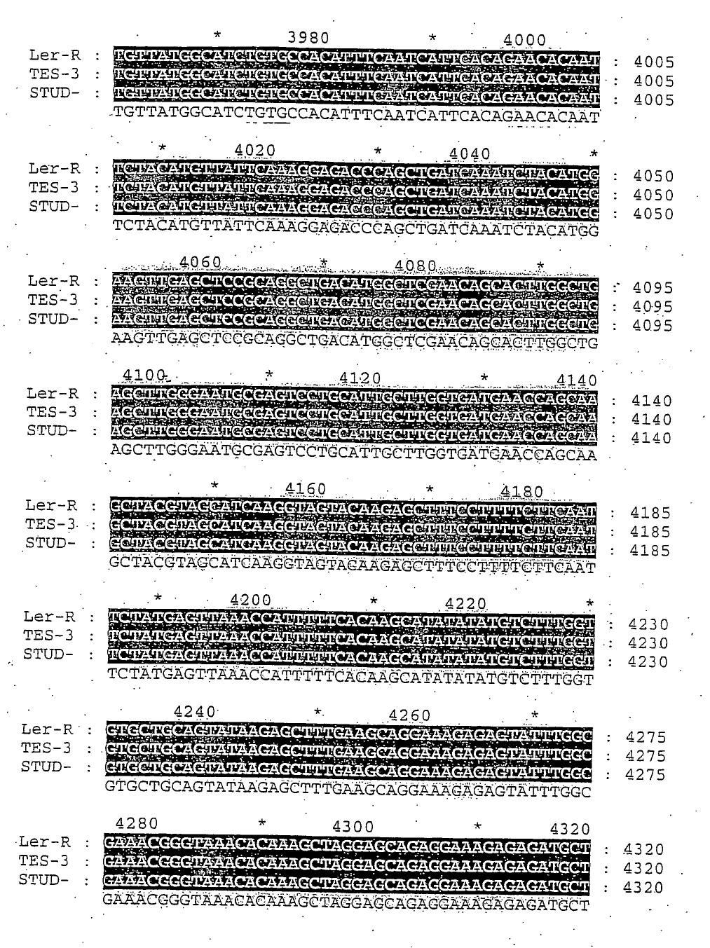

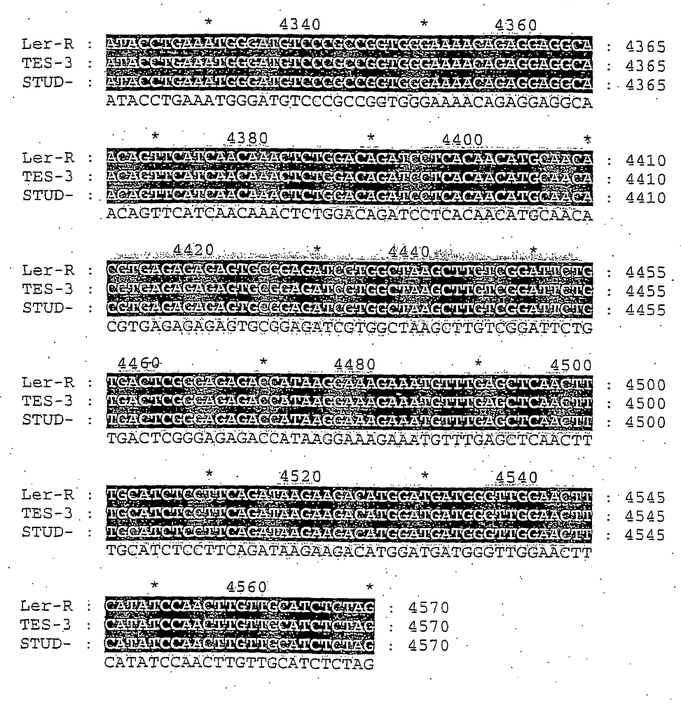

Database searches have identified a very similar protein to TES on chromosome 1 of Arabidopsis and a homologous rice kinesin sequence (see FIG. 3). Phylogenetic analysis reveals TES and the two homologous kinesin forming a distinct group.

4. Expression Analysis of tes

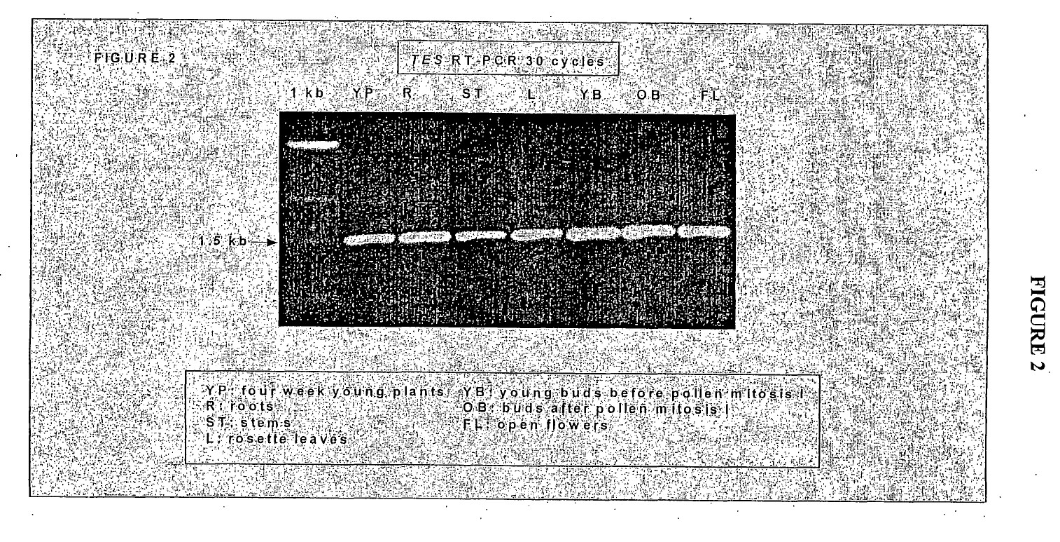

RT-PCR expression analysis showed TES to be expressed in all parts of the Arabidopsis plant (FIG. 2).

1. A plant in which, by virtue of modulation of the expression of a TETRASPORE (TES) or TES-like (TLK) gene, or of modulation of the activity of a protein encoded by such a gene, tetrad formation in the anther is disrupted, or is capable of being disrupted, such that callose cross-wall formation in the tetrad fails to the extent that fusion between two or more of the four microspore nuclei may occur.

2. A plant according to claim 1 wherein fusion between two or more of the tetrad nuclei results in the formation of sperm of greater than half the ploidy of its somatic cells.

3. A plant according to claim 1 or 2 wherein fusion between the nuclei of the tetrad results in the formation of sperm of heterogeneous ploidy.

4. A plant according to claim 2 or 3 wherein at least some of said sperm are diploid or triploid.

5. A plant according to any one of the preceding claims wherein modulation of said TES or TLK gene is by virtue of a transgenic construct which provides for expression of antisense RNA or RNAi that suppresses the TES or TLK gene.

6. A plant according to any one of the preceding claims wherein modulation of said TES or TLK gene is inducible.

7. A plant according to claim 6 wherein modulation is inducible by virtue of a transgenic construct as defined in claim 5 wherein expression of the antisense RNA or RNAi is under the control of an inducible promoter.

8. A plant according to any one of the preceding claims wherein said TES or TLK gene:
(a) encodes a polypeptide sequence as set out in SEQ ID NO: 1, 2 or 3; or
(b) has a cDNA sequence having 60% or more homology to a sequence of (a); or
(c) has a cDNA sequence capable of hybridising selectively to the complement of a sequence of (a); and
which gene, when functional, encodes a kinesin protein; and, when non-functional, causes the plant to fail to form callose cross-walls in the tetrad.

9. A plant according to any one of the preceding claims which is an ornamental plant, or an arable crop plant.

10. A progeny plant of a plant of any one of the preceding claims.

11. A method of obtaining a progeny plant from male and female parent plants whose somatic cells have different genome ploidy, said method comprising (a) providing as a male parent a plant according to any one of claims 1 to 9; (b) providing as a female parent a plant whose ploidy differs from the plant of (a); (c) allowing the male parent of (a) to produce pollen containing sperm of greater than half the ploidy of its somatic cells, and optionally of heterogeneous ploidy; (d) allowing the male parent of (a) to pollinate the female parent of (b); and recovering a seed that results from said pollination; and optionally (e) growing up a progeny plant from said seed.

12. A method of obtaining a progeny plant from male and female parent plants whose somatic cells have different genome ploidy, said method comprising producing a plant according to any one of claims 1 to 9; and (a) providing said plant as a male parent plant; (b) providing as the female parent a plant whose ploidy differs from the plant of (a); (c) allowing the male parent of (a) to produce pollen containing sperm of greater than half the ploidy of its somatic cells, and optionally of heterogeneous ploidy; (d) allowing the male parent of (a) to pollinate the female parent of (b); and recovering a seed that results from said pollination; and optionally (e) growing up a progeny plant from said seed.

13. A method according to claim 12 wherein said male parent plant is produced by transformation with a construct according to claim 5, or by inactivating a TES or TLK gene by mutation.

14. A method according to claim 12 or 13 wherein the male and female parent plants are of:
(i) different varieties, subspecies, lines or cultivars within the same species; or
(ii) different species within the same genus; or
(iii) different species in different genera.

15. Use of a plant according to any one of claims 1 to 9 in a breeding programme.

16. An TES polynucleotide which:
(a) encodes a polypeptide sequence as set out in SEQ ID NO: 1; or
(b) has a cDNA sequence having 60% or more homology to a sequence of (a); or
(c) has a cDNA sequence capable of hybridising selectively to the complement of a sequence of (a); and
encodes a kinesin

17. A polynucleotide sequence capable of hybridising selectively to all or part of the mRNA expressed from a gene as defined in claim 8 and thus achieving antisense inhibition of said gene.

18. A vector comprising a polynucleotide according to claim 16 or 17 operably linked to a promoter.

19. A vector according to claim 18 wherein the promoter is stage-specific and/or tissue specific and/or inducible.

20. A vector according to claim 18 or 19 wherein the polynucleotide is an antisense polynucleotide according to claim 17 and the promoter is an inducible promoter.

21. A cell transformed with a polynucleotide sequence according to claim 16 or 20 or a vector according to claim 18, 19 or 20.

22. A cell according to claim 21 which is a plant cell.

23. A method of making a cell according to claim 21 or 22 comprising transforming a cell with a polynucleotide sequence according to claim 16 or 17 or a vector according to claim 18, 19 or 20.

24. A method according to claim 23 further comprising regenerating a plant from said transformed cell, and optionally further propagating said plant to give progeny plants of first, second or subsequent generations.

25. A polypeptide encoded by a polynucleotide according to claim 16.

26. A polypeptide according to claim 25 having the sequence of SEQ ID NO: 1.

27. An antibody which specifically recognises a polypeptide according to claim 25 or 26.

28. A primer, or pair of primers, capable of amplifying a polynucleotide as defined in claim 16 or 17 in a Polymerase Chain Reaction.

29. A labelled probe capable of selectively hybridising to a polynucleotide as defined in claim 16 or 17 and thus detecting said polynucleotide.


SEQUENCE LISTING (10 sequences)

SEQ ID NO: 1 - protein, 932 aa, Arabidopsis thaliana
MGPPRTPLSKIDKSNPYTPCGSKVTEEKILVTVRMRPLNWREHAKYDLIAWECPDDETIVFKNPNPDKAPTKYSFDKVFEPTCATQEVYEGGSRDVALSALAGTNATIFAYGQTSSGKTFTMRGVTESVVKDIYEHIRKTQERSFVLKVSALEIYNETVVDLLNRDTGPLRLLDDPEKGTIVENLVEEVVESRQHLQHLISICEDQRQVGETALNDKSSRSHQIIRLTIHSSLREIAGCVQSFMATLNLVDLAGSERAFQTNADGLRLKEGSHINRSLLTLTTVIRKLSSGRKRDHVPYRDSKLTRILQNSLGGNARTAIICTISPALSHVEQTKKTLSFAMSAKEVTNCAKVNMVVSEKKLLKHLQQKVAKLESELRSPEPSSSTCLKSLLIEKEMKIQQMESEMKELKRQRDIAQSELDLERKAKERKGSSECEPFSQVARCLSYHTKEESIPSKSVPSSRRTARDRRKDNVRQSLTSADPTALVQEIRLLEKHQKKLGEEANQALDLIHKEVTSHKLGDQQAAEKVAKMLSEIRDMQKSNLLTEEIVVGDKANLKEEINRLNSQEIAALEKKLECVQNTIDMLVSSFQTDEQTPDFRTQVKKKRLLPFGLSNSPNLQHMIRGPCSPLSGTENKDPESNVVSANSAPVSFGATPPKRDDNRCRTQSREGTPVSRQANSVDIKRMNRMYKNAAEENIRNIKSYVTGLKERVAKLQYQKQLLVCQANETGAASEYDATDESQMDWPLCFEEQRKQIIMLWHLCHISIIHRTQFYMLFKGDPADQIYMEVELRRLTWLEQHLAELGNASPALLGDEPASYVASSIRALKQEREYLAKRVNTKLGAEEREMLYLKWDVPPVGKQRRQQFINKLWTDPHNMQHVRESAEIVAKLVGFCDSGETIRKEMFELNFASPSDKKTWMMGWNFISNLLHL

SEQ ID NO: 2 - protein, 954 aa, Oryza sativa
MGVSRPPSTPASKIERTPMSTPTPGGSTRVKEEKIFVTVRVRPLSKKELALKDQVAWECDDNQTILYKGPPQDRAAPTSYTFDKVFGPASQTEVVYEEGAKDVAMSALTGINATIFAYGQTSSGKTFTMRGVTESAVNDIYRHIENTPERDFIIKISAMEIYNEIVKDLLRPESTNLRLLDDPEKGTIVEKLEEEIAKDSQHLRHLISICEEQRQVGETALNDTSSRSHQIIRLTVESRLREVSGCVKSFVANLNFVDLAGSERAAQTHAVGARLKEGCHINRSLLTLTTVIRKLSSDKRSGHIPYRDSKLTRILQLSLGGNARTAIICTMSPAQTHVEQSRNTLFFATCAKEVTNNAKVNMVVSDKQLVKHLQMEVARLEAELRTPDRASSSEIIIMERDRKIRQMEKEMEELKKQRDNAQLKLEELQKKMGDNQPGWNPFDSPQRTRKCLTYSGSLQPSNKMKIRSSIRQSATAPFMLKHEIRKLEQLQQQLEVEANRAIEVLHKEVECHKHGNQDAAETIAKLQAEIRGMQSVRSDRDVDMITDEGNGSDLKEEISRLHMQDNDIAKLEAKLENVQRSIDRLVMSLPNVGTQCNETTPKSNRAKKKKRMLLPLGVSNINRPNLIRAPCSPLSSSRPLEPEVENRAPEGDTVSHEGSERATPTKSEDTGDVSSRDETPRYRRSSSVNMKKMQKMFQNAAEENVRNIRAYVTELKERVAKLQYQKQLLVCQVLELESNEGKTNDMEEDSEENAGSLQDGPDSWDRLFKEQMQHIIQLWDLCHVSIIHRTQFYLLFRGDRADQIYIEVEVRRLTWLQQHFAEVGDASPAAGDDSTISLASSIKALRNEREFLARRMGSRLTEEERERLFIKWQVPLEAKQRKLQLVNRLWTDPNDQAHIDESADIVARLVGFCEGGNISKEMFELNFAVPASRKPWLMGWQPISNMIREKTQLW

SEQ ID NO: 3 - protein, 1004 aa, Arabidopsis thaliana
MTIKTPGTPVSKMDRTPAVTPGGSSRSREEKIVVTVRLRPMNKRELLAKDQVAWECVNDHTIVSKPQVQERLHHQSSFTFDKVFGPESLTENVYEDGVKNVALSALMGINATIFAYGQTSSGKTYTMRGVTEKAVNDIYNHIIKVSTALVRVNVLDVKCSSSDQYLYLFHCCFQTPERDFTIKISGLEIYNENVRDLLNSDSGRALKLLDDPEKGTVVEKLVEETANNDNHLRHLISICEAQRQVGETALNDTSSRSHQIIRLNFVDLAGSERASQSQADGTRLREGCHINLSLMTLTTVIRKLRYCTYIFSERLKSQSQILFNVGKRSGHIPYRDSKLTRILQHSLGGNARTAIICTLSPALAHVEQSRNTLYFANRAKEVTNNAHVNMVVSDKQLVKHLQKEVARLEAERRTPGPSTEKDFKIQQMEMEIGELRRQRDDAQIQLEELRQKLQQDQQQNKGLNPFESPDPPVRKCLSYSVAVTPSSENKRLNRNERARKTTMRQSMIRQSSTAPFTLMHEIRKLEHLQEQLGEEATKALEVLQKEVACHRLGNQDAAQTIAKLQAEIREMRTVKPSAMLKEVGDVIAPNKSVSANLKEEITRLHSQGSTIANLEEQLESVQKSIDKLVMSLPSNISAGDETPKTKNHHHQSKKKKLLPLTPASASNRQNFLKSPCSPLSASRQVLDCDAENKAPQENNSSAARGATTPQGSEKETPQKGEESGDVSSREGTPGYRRSSSVNMKKMQQMFQNAAEENVRSIRAYVTELKERVAKLQYQKQLLVCQVLELEANDGAGYSVENEENTIMEDEEQNQVAWHITFIEERQQIIELWHVCHVSIIHRTQFYLLFKGDQADGQIYMEVELRRLTWLEQHLAEVGNATPARNCDESVVSLSSSIKALRREREFLAKRVNSRLTPEEREELYMKWDVPLEGKQRKLQFVNKLWTDPYDSRHVQESAEIVAKLVGFCESGNISKEMFELNFAVPSDKRQWNIGWDNISNLLHL

SEQ ID NO: 4 - DNA, 4590 bp, Arabidopsis thaliana
atgggacctccgagaactccgttaagtaagatagataagtccaatccttatactccatgtggttccaaagttacagaggagaagattcttgtcactgttcggatgagaccattaaattggagggaacatgccaaatatgatctcattgcttgggaatgtcccgatgatgagactatcgtctttaagaatccgaatcctgacaaggctccaacaaaatattcatttggtattttttattagaatctagtcgattgcttattttgtcttctcttctttccatttttgttatatagctcatttaagttttttcttctttgtacttatagataaagtttttgagccaacttgtgcaactcaagaggtgtatgaaggtggttctagggacgttgcactatctgcattagcaggaacaaatggtaactacacagaaatctaaaaaatcccattatctaaggatatgttatttgtggatatgtgatttcttgtctctatctattgcagcaactatttttgcttatggtcagactagtagtggcaagacgtttacaatgaggggggttactgaaagtgttgttaaagatatatacgaacatataagaaaagtgagtgatcaaatggatgtgttttggttttggttttgttacgaagtgatatgttctttttctattctcatggatctgttgaaatctatgtagactcaagaaaggagctttgtcttgaaagtatctgctttggagatttataacgagaccgtggttgaccttttaaatcgtgatactgggccccttagactgttagatgatccagaggtaagtaaagggtcggaaacactaagtcaaggctcatgaactatctcggccagtataagaatgctgtgtatattgcagaaaggaaccattgtggaaaatctggtggaggaagtggtagaaagcaggcaacatttacagcatttaattagcatttgtgaaggtaattcttctatcacctgttaagatgctctatatctttttatgcatttgatggtgatagtattctaagtaatgtttagcaaagccttagaaccatgtagatgacatatataaaatatgactaattatttcactaatcactaatgagacatatgatatcatcattttatttcctgttatgtgattttccatttattgttacagatcaaaggcaagtaggtgaaactgctttaaacgacaaaagttcaagatcgcatcagattattcggctggtatgatctatacttatctgatatgctgcatgattgaattaatattctattaaacatggagaagatagttttgcctagttatttgctaccattagacagtaaaagtaaccacttcccataatttctcatgttctctgcttttaatattcagaccatccatagcagtctcagggaaatagcaggctgtgtgcagtctttcatggcaactctggtacgtcatttattatcattgggttgaagcttcacttggtattgacgacattttaattggtgcagaatcttgttgatcttgctggaagcgaacgtgcttttcaaacaaacgcagatggtctaagattgaaggaaggaagccacattaaccgtagtttattgacacttactacagtgatcagaaaactgaggtcagaaagcacactagtttgaacaccatgattgtctttggcatgtctaaattaccttttagattgatgaacattttcacctgtagggcatcttaagaaattagcaaaacattaaagtttttaattatttctgttattttggttgaagttctggaagaaaaagggatcacgtgccgtatagagactcaaagctgacaagaattttgcaaaattcacttggcgggaatgctagaacagcaattatatgtacgattagtccggcactgagtcatgttgaacagacaaaaaagacactttcttttgccatgagtgcgaaggaagtcaccaattgcgctaaagtaaacatggtatgtcacttagttaaatatttgtagaggtctttcgttttgcagaaccaggatgattgtatagagctttgtctttggcttactaaagcttttctttggcctttcattgcaaatgtggttttgtttctgagtagcaatcatcatcctatagtgagcatatcatatgcaattatctgcctgcatgtctattctgctcttgccatgaattaagaatacaaaatgcatcgcgttacctgtaatgatgtagtgagatatattttcaaagccttcttttgtcgctctcccactaacattactccttttgcttcaatcttctttgtgtttgatgaatcacaaaacggttttgcttgttcttgatagaaagtttacccctctgctggtattcaatttagggatgtgaatttagttgattttctgtcgtaggttgtctcagaaaaaaagctgctgaaacatctgcagcaaaaagtagctaaactcgagtcagagttaagaagcccggagcccagttcatccacctgcttaaagtctctgttaattgagaaagagatgaaaatccaacaggtatgacttagactttaagaactagaattcacatcttgaattatctcttctgaatattttaaatgtggagtccacagactaggtaaaccatatgggacgtgaacttaagtttattaacaaaaacaacttgaattttttatcacttgcaattattcattcatgtttaactaatggcagatggaatccgaaatgaaagagctaaagcgtcagagagacattgcacagtctgaacttgatctagaaagaaaagcaaaggagcggaaggtatgtgcgctgctaatatgatataccaaaattttctgggataacttcaaccttatgcagacatgcctatataattggttgcagggatcaagtgagtgtgaacctttttctcaagtggcaaggtgtctctcataccatacaaaggaggaatccattccaagcaaatctgttccgtcaagccgcagaacagctcgtgataggagaaaggataatgtgaggcagtctttaacttcggcggatccaacagcgctagtccaagaaattcgtttgcttgaaaaacatcaaaagaagttgggtgaagaagcaaaccaagcactggatctaattcacaaagaagtgacatcccataaactgggtgatcaacaagctgcagaaaaagttgcgaaaatgctgtcagaaatcagggacatgcagaagagcaaccttttaactgaggaaattgtagttggtgataaagccaacctgaaggaagaaataaatcggttgaattctcaagaaattgcagctttggagaagaagctagagtgtgttcagaacactattgacatgcttgtctcgtcttttcagacagatgaacagactccagatttcaggacccaggtgaaaaagaagagacttcttcccttcggcttaagtaatagtcctaacctacagcatatgatccggggaccatgttcacctctttctgggactgaaaataaggatcctgagagcaatgttgtgtctgctaattcagccccagtgtcttttggagctactccaccaaagagggatgataaccgatgtcgtacacagtcaagggaaggaacgcctgtatcacgacaagcaaattcagtcgatattaagagaatgaatcgaatgtataagaatgctgccgaagagaatatacgtaatatcaaatcttatgttactggattgaaagaacgtgtggcgaagcttcaataccagaagcagctgcttgtttgccaggtcagtttacacttgtgacttatgctcattcatttacaaatttgttcatcagtaatcagtaacttaaaaagcttgtacatgttaggtgttagaactggaggcaaacgagacaggcgctgcttcggagtacgatgcaaccgatgaatctcagatggattggccattgtgttttgaagagcagagaaagcaaatcataatgttatggcatctgtgccacatttcaatcattcacagaacacaattctacatgttattcaaaggagacccagctgatcaaatctacatggaggttgagctccgcaggctgacatggctcgaacagcacttggctgagcttgggaatgcgagtcctgcattgcttggtgatgaaccagcaagctacgtagcatcgaggtagtacaagagctttccttttcttcaattctatgagttaaaaccatttttcactagcatatatatgtctttggtgtgctgcagtataagagctttgaagcaggagagagagtatttggcgaaacgggtaaacacaaagctaggagcagaggaaagagagatgctatacctgaaatgggatgtcccgccggtgggaaaacagaggagacaacagttcatcaacaaactctggacagatcctcacaacatgcaacacgtgagagagagtgcggagatcgtggctaagcttgtcggattctgtgactcgggagagaccataaggaaagaaatgtttgagctcaactttgcatctccttcagataagaagacttggatgatgggttggaacttcatatccaacttgttgcatctctag

SEQ ID NO: 5 - DNA, 4583 bp, Artificial sequence
  TES-1 MUTANT SEQUENCE
atgggacctccgagaactccgttaagtaagatagataagtccaatccttatactccatgtggttccaaagttacagaggagaagattcttgtcactgttcggatgagaccattaaattggagggaacatgccaaatatgatctcattgcttgggaatgtcccgatgatgagactatcgtctttaagaatccgaatcctgacaaggctccaacaaaatattcatttggtattttttattagaatctagtcgattgcttattttgtcttctcttctttccatttttgttatatagctcatttaagttttttcttctttgtacttatagataaagtttttgagccaacttgtgcaactcaagaggtgtatgaaggtggttctagggacgttgcactatctgcattagcaggaacaaatggtaactacacagaaatctaaaaaatcccattatctaaggatatgttatttgtggatatgtgatttcttgtctctatctattgcagcaactatttttgcttatggtcagactagtagtggcaagacgtttacaatgaggggggttactgaaagtgttgttaaagatatatacgaacatataagaaaagtgagtgatcaaatggatgtgttttggttttggttttgttacgaagtgatatgttctttttctattctcatggatctgttgaaatctatgtagactcaagaaaggagctttgtcttgaaagtatctgctttggagatttataacgagaccgtggttgaccttttaaatcgtgatactgggccccttagactgttagatgatccagaggtaagtaaagggtcggaaacactaagtcaaggctcatgaactatctcggccagtataagaatgctgtgtatattgcagaaaggaaccattgtggaaaatctggtggaggaagtggtagaaagcaggcaacatttacagcatttaattagcatttgtgaaggtaattcttctatcacctgttaagatgctctatatctttttatgcatttgatggtgatagtattctaagtaatgtttagcaaagccttagaaccatgtagatgacatatataaaatatgactaattatttcactaatcactaatgagacatatgatatcatcattttatttcctgttatgtgattttccatttattgttacagatcaaaggcaagtaggtgaaactgctttaaacgacaaaagttcaagatcgcatcagattattcggctggtatgatctatacttatctgatatgctgcatgattgaattaatattctattaaacatggagaagatagttttgcctagttatttgctaccattagacagtaaaagtaaccacttcccataatttctcatgttctctgcttttaatattcagaccatccatagcagtctcagggaaatagcaggctgtgtgcagtctttcatggcaactctggtacgtcatttattatcattgggttgaagcttcacttggtattgacgacattttaattggtgcagaatcttgttgatcttgctggaagcgaacgtgcttttcaaacaaacgcagatggtctaagattgaaggaaggaagccacattaaccgtagtttattgacacttactacagtgatcagaaaactgaggtcagaaagcacactagtttgaacaccatgattgtctttggcatgtctaaattaccttttagattgatgaacattttcacctgtagggcatcttaagaaattagcaaaacattaaagtttttaattatttctgttattttggttgaagttctggaagaaaaagggatcacgtgccgtatagagactcaaagctgacaagaattttgcaaaattcacttggcgggaatgctagaacagcaattatatgtacgattagtccggcactgagtcatgttgaacagacaaaaaagacactttcttttgccatgagtgcgaaggaagtcaccaattgcgctaaagtaaacatggtatgtcacttagttaaatatttgtagaggtctttcgttttgcagaaccaggatgattgtatagagctttgtctttggcttactaaagcttttctttggcctttcattgcaaatgtggttttgtttctgagtagcaatcatcatcctatagtgagcatatcatatgcaattatctgcctgcatgtctattctgctcttgccatgaattaagaatacaaaatgcatcgcgttacctgtaatgatgtagtgagatatattttcaaagccttcttttgtcgctctcccactaacattactccttttgcttcaatcttctttgtgtttgatgaatcacaaaacggttttgcttgttcttgatagaaagtttacccctctgctggtattcaatttagggatgtgaatttagttgattttctgtcgtaggttgtctcagaaaaaaagctgctgaaacatctgcagcaaaaagtagctaaactcgagtcagagttaagaagcccggagcccagttcatccacctgcttaaagtctctgttaattgagaaagagatgaaaatccaacaggtatgacttagactttaagaactagaattcacatcttgaattatctcttctgaatattttaaatgtggagtccacagactaggtaaaccatatgggacgtgaacttaagtttattaacaaaaacaacttgaattttttatcacttgcaattattcattcatgtttaactaatggcagatggaatccgaaatgaaagagctaaagcgtcagagagacattgcacagtctgaacttgatctagaaagaaaagcaaaggagcggaaggtatgtgcgctgctaatatgatataccaaaattttctgggataacttcaaccttatgcagacatgcctatataattggttgcagggatcaagtgagtgtgaacctttttctcaagtggcaaggtgtctctcataccatacaaaggaggaatccattccaagcaaatctgttccgtcaagccgcagaacagctcgtgataggagaaaggataatgtgaggcagtctttaacttcggcggatccaacagcgctagtccaagaaattcgtttgcttgaaaaacatcaaaagaagttgggtgaagaagcaaaccaagcactggatctaattcacaaagaagtgacatcccataaactgggtgatcaacaagctgcagaaaaagttgcgaaaatgctgtcagaaatcagggacatgcagaagagcaaccttttaactgaggaaattgtagttggtgataaagccaacctgaaggaagaaataaatcggttgaattctcaagaaattgcagctttggagaagaagctagagtgtgttcagaacactattgacatgcttgtctcgtcttttcagacagatgaacagactccagatttcaggacccaggtgaaaaagaagagacttcttcccttcggcttaagtaatagtcctaacctacagcatatgatccggggaccatgttcacctctttctgggactgaaaataaggatcctgagagcaatgttgtgtctgctaattcagccccagtgtcttttggagctactccaccaaagagggatgataaccgatgtcgtacacagtcaagggaaggaacgcctgtatcacgacaagcaaattcagtcgatattaagagaatgaatcgaatgtataagaatgctgccgaagagaatatacgtaatatcaaatcttatgttactggattgaaagaacgtgtggcgaagcttcaataccagaagcagctgcttgtttgccaggtcagtttacacttgtgacttatgctcattcatttacaaatttgttcatcagtaatcagtaacttaaaaagcttgtacatgttaggtgttagaactggaggcaaacgagacaggcgctgcttcggagtacgatgcaaccgatgaatctcagatggattggccattgtgttttgaagagcagagaaagcaaatcataatgttatggcatctgtgccacatttcaatcattcacagaacacaattctacatgttattcaaaggagacccagctgatcaaatctacatggaggttgagctccgcaggctgacatggctcgaacagcacttggctgagcttgggaatgcgagtcctgcattgcttggtgatgaaccagcaagctacgtagcatcgaggtagtacaagagctttccttttcttcaattctatgagttaaaaccatttttcactagcatatatatgtctttggtgtgctgcagtataagagctttgaagcaggagagagagtatttggcgaaacgggtaaacacaaagctaggagcagaggaaagagagatgctatacctgaaatgggatgtcccgccggtgggaaaacagaggagacaacagttcatcaacaaactctggacagatcctcacaacatgcaacacgtgagagagagtgcggagatcgtggctaagcttgtcggattctgtgactcgggagagaccataaggaaagaaatgtcaactttgcatctccttcagataagaagacttggatgatgggttggaacttcatatccaacttgttgcatctctag

SEQ ID NO: 6 - DNA, 4570 bp, Artificial sequence
  WS2.R TES SEQUENCE
atgggacctccgagaactccgttaagtaagatagataagtccaatccttatactccatgtggttccaaagttacagaggagaagattcttgtcactgttcggatgagaccattaaattggagggaacatgccaaatatgatctcattgcttgggaatgtcctgatgatgaaactatcgtctttaagaatccgaatcctgacaaggctccaacaaaatattcatttggtatttttattagaatctagtcaattgcttatttgtcttctcttctttccaattttgttatatagctcatttaagtttttgtcttgtttgtacttacagataaagtttttgagccaacttgtgccactcaagaggtgtatgaaggtggttctagggacgttgcactatctgcattagcaggaacaaatggtaaatacacagatatctaaaaaaatcccattatctaaggatattttatttgtggatatgtgatttcttgtctctatctattgcagcaactatttttgcttatggtcagactagtagtggcaagacgtttacaatgaggggggttacggaaagtgttgttaaagatatatacgaacatataagaaaagtgagtgaccaaatggatgtgttttggttttggttttgttacgaagtgatatgttctttttctatactcatggatctgttgaaatctatgtagactcaagaaaggagctttgtcttgaaagtatctgctttggagatctataatgagaccgtggttgaccttttaaatcgtgatactgggccccttcgactgttagatgatccagaggtaagtaaagggtcggaaacactaggctcatgaactatctcggccagtataagaatgttgtgtatgttgcagaaaggaaccattgtggaaaatctggtggaggaagtggtagaaagcaggcaacatttacagcatttaattagcatttgtgaaggtaattcttctatcacctgttaagatgctctatatctctttatgcatttgatgatgatagtattctaagtcatgtttagcaaagccttagaaccatgtagatgacatatataaaatatgactcattatttcactaatcactaatgagacatatgatatcatctttttatttcctgttatgtgattttccatttattgtacagatcaaaggcaagtaggtgaaactgctttaaacgacaaaagttcaagatcgcatcagattattcggctggtatgatctatactcatctgatatgctgcataattgaattaatattctattaaacatggagaagatagttttgcctagttatttgctaccattagacagtaaaagtaaccactttccataatttctcatgttctctgcttttaatattcagaccatccatagcagtctcagggaaatagcaggctgtgtgcagtctttcatggcaactctggtacgtcatttattatcatagggttgaagcttcacttggtattgacgacattttaattggtgcagaatcttgttgatcttgctggaagcgaacgtgcttttcaaacaaacgcagatggtctaagactgaaggaaggaagccacattaaccgtagtttattgacacttactacagtgatcagaaaactgaggtcagaaagcacactagtctgaacaccatgattatctttggcatgtctaaattaccttttaggttgatgaacattttcacccgtagggcaccttaagaaattagcaaaacatttaattatttctgttattttggtgaagttctggaagaaaaagggatcacgtgccatatagagactcaaagctgacaagaattttgcaaaattcacttggcgggaatgctagaacagcaattatctgtacgattagcccagcactgagtcacgttgaacagacaaaaaagacactctcttttgccatgagtgcgaaggaagtcaccaactgcgctaaagtaaacatggtatgtcacttagttaaatatttggagaggtctttcgttttgcagaaacaggatgattgtttggagctttgtctttggcttcctaaagcttttctttggtctttcattgaaaatgtggtttgtttgtgagtagcaatcatcatcctatagtgagcatatcatatgcaattatctgcctgcatagctattctgctcttcccatgaattaaggatacaaaatgcatcgcgttacctgtaatgatgtagtaagatataattcaaagccttctttgtcgctctcccactaacattactccttttgcttcaatcttctttgtgtttgatgaataacaaaacggttttgcttgttcttgatagaaagtttacccctctactggtattcaatttagggatgtgaatttagttgattttctgttgtaggttgtctcagaaaaaaagctgctgaaacatctgcagcaaaaagtagctaaactcgagtcagagttaagaagcccggagcccagttcatccacctgcttaaagtctctgttaattgagaaagagatgaaaatccaacaggtatgacttagactttaagaactagaattcacatcttgaattatctcttctgaatattttaaatgtggagtccacagactaggtaaaccatatgggacgtgaacttaagtttattaacaaaaacaacttgaattttttatgacttgcaattattcattcatgtttaactaatggcagatggaatccgaaatgaaagagctaaagcgtcagagagacattgcacagtctgaacttgatctagaaagaaaagcaaaggagcggaaggtatgtgcgctgctaatatgatataccaaaattttctgggataacttcaaccttatgcagacatgcctatataattggttgcagggatcaagtgagtgtgaacctttttctcaagtggcaaggtgtctctcataccatacaaaggaggaatccattccaagcaaatctgttccgtcaagccgcagaacagctcgtgataggagaaaggataatgtgaggcagtctttaacttcagcggatccaacagcgctagtccaagaaattcgtttgcttgaaaaacatcaaaagaagttgggtgaagaagcaaaccaagcactggatctaattcacaaagaagtgacatcccataaactgggtgatcaacaagctgcagagaaagttgcgaaaatgctatcagaaatcagggacatgcagaagagcaaccttttaactgaggaaattgtagttggtgataaagctaacctgaaggaagaaataaatcgtttgaattctcaagaaattgcagctttggagaagaagctagagtgtgttcagaacactattgacatgcttgtctcgtcttttcagacagatgaacagactccagatttcaggacccaggtgaaaaagaagagacttcttcccttcggcttaagtaatagtcctaacctacagcatatgatccggggaccatgttcacctctttctgggactgaaaataaggatcctgagagcaatgttgtgtctgctaattcaatcccagtgtcttttggagctactccaccaaagagggatgataaccgatgtcgtacaccgtcaagggaaggaacgcctgtatcacgacaagcaaattcagtcgatattaagagaatgaatcgaatgtataagaatgctgccgaagaaaatatacgtaacatcaaatcttatgttactggattgaaagaacgtgtggcgaagcttcaataccagaagcagcttcttgtttgccaggtcagtttacacttgtgacttatgctcattcatttacaaatttgttcatcaggaatcagtaacttaaaagcttgcacatgtataggtgttagaactggaggcaaacgagacaggcgctgcttcggagtacgatgcaaccgatgaatctcggatggactggccattgtgttttgaagagcagagaaagcaaatcataatgttatggcatctgtgccacatttcaatcattcacagaacacaattctacatgttattcaaaggagacccagctgatcaaatctacatggaagttgagctccgcaggctgacatggctcgaacagcacttggctgagcttgggaatgcgagtcctgcattgcttggtgatgaaccagcaagctacgtagcatcaaggtagtacaagagctttccttttcttcaattctatgagttaaaccatttttcacaagcatatatatgtctttggtgtgctgcagtataagagctttgaagcaggaaagagagtatttggcgaaacgggtaaacacaaagctaggagcagaggaaagagagatgctatacctgaaatgggatgtcccgccggtgggaaaacagaggaggcaacagttcatcaacaaactctggacagatcctcacaacatgcaacacgtgagagagagtgcggagatcgtggctaagcttgtcggattctgtgactcgggagagaccataaggaaagaaatgtttgagctcaactttgcatctccttcagataagaagacatggatgatgggttggaacttcatatccaacttgttgcatctctag

SEQ ID NO: 7 - DNA, 4560 bp, Artificial sequence
  TES-4 MUTANT SEQUENCE
atgggacctccgagaactccgttaagtaagatagataagtccaatccttatactccatgtggttccaaagttacagaggagaagattcttgtcactgttcggatgagaccattaaattggagggaacatgccaaatatgatctcattgcttgggaatgtcctgatgatgaaactatcgtctttaagaatccgaatcctgacaaggctccaacaaaatattcatttggtatttttattagaatctagtcaattgcttatttgtcttctcttctttccaattttgttatatagctcatttaagtttttgtcttgtttgtacttacagataaagtttttgagccaacttgtgccactcaagaggtgtatgaaggtggttctagggacgttgcactatctgcattagcaggaacaaatggtaaatacacagatatctaaaaaaatcccattatctaaggatattttatttgtggatatgtgatttcttgtctctatctattgcagcaactatttttgcttatggtcagactagtagtggcaagacgtttacaatgaggggggttacggaaagtgttgttaaagatatatacgaacatataagaaaagtgagtgaccaaatggatgtgttttggttttggttttgttacgaagtgatatgttctttttctatactcatggatctgttgaaatctatgtagactcaagaaaggagctttgtcttgaaagtatctgctttggagatctataatgagaccgtggttgaccttttaaatcgtgatactgggccccttcgactgttagatgatccagaggtaagtaaagggtcggaaacactaggctcatgaactatctcggccagtataagaatgttgtgtatgttgcagaaaggaaccattgtggaaaatctggtggaggaagtggtagaaagcaggcaacatttacagcatttaattagcatttgtgaaggtaattcttctatcacctgttaagatgctctatatctctttatgcatttgatgatgatagtattctaagtcatgtttagcaaagccttagaaccatgtagatgacatatataaaatatgactcattatttcactaatcactaatgagacatatgatatcatctttttatttcctgttatgtgattttccatttattgtacagatcaaaggcaagtaggtgaaactgctttaaacgacaaaagttcaagatcgcatcagattattcggctggtatgatctatactcatctgatatgctgcataattgaattaatattctattaaacatggagaagatagttttgcctagttatttgctaccattagacagtaaaagtaaccactttccataatttctcatgttctctgcttttaatattcagaccatccatagcagtctcagggaaatagcaggctgtgtgcagtctttcatggcaactctggtacgtcatttattatcatagggttgaagcttcacttggtattgacgacattttaattggtgcagaatcttgttgatcttgctggaagcgaacgtgcttttcaaacaaacgcagatggtctaagactgaaggaaggaagccacattaaccgtagtttattgacacttactacagtgatcagaaaactgaggtcagaaagcacactagtctgaacaccatgattatctttggcatgtctaaattaccttttaggttgatgaacattttcacccgtagggcaccttaagaaattagcaaaacatttaattatttctgttattttggtgaagttctggaagaaaaagggatcacgtgccatatagagactcaaagctgacaagaattttgcaaaattcacttggcgggaatgctagaacagcaattatctgtacgattagcccagcactgagtcacgttgaacagacaaaaaagacactctcttttgccatgagtgcgaaggaagtcaccaactgcgctaaagtaaacatggtatgtcacttagttaaatatttggagaggtctttcgttttgcagaaacaggatgattgtttggagctttgtctttggcttcctaaagcttttctttggtctttcattgaaaatgtggtttgtttgtgagtagcaatcatcatcctatagtgagcatatcatatgcaattatctgcctgcatagctattctgctcttcccatgaattaaggatacaaaatgcatcgcgttacctgtaatgatgtagtaagatataattcaaagccttctttgtcgctctcccactaacattactccttttgcttcaatcttctttgtgtttgatgaataacaaaacggttttgcttgttcttgatagaaagtttacccctctactggtattcaatttagggatgtgaatttagttgattttctgttgtaggttgtctcagaaaaaaagctgctgaaacatctgcagcaaaaagtagctaaactcgagtcagagttaagaagcccggagcccagttcatccacctgcttaaagtctctgttaattgagaaagagatgaaaatccaacaggtatgacttagactttaagaactagaattcacatcttgaattatctcttctgaatattttaaatgtggagtccacagactaggtaaaccatatgggacgtgaacttaagtttattaacaaaaacaacttgaattttttatgacttgcaattattcattcatgtttaactaatggcagatggaatccgaaatgaaagagctaaagcgtcagagagacattgcacagtctgaacttgatctagaaagaaaagcaaaggagcggaaggtatgtgcgctgctaatatgatataccaaaattttctgggataacttcaaccttatgcagacatgcctatataattggttgcagggatcaagtgagtgtgaacctttttctcaagtggcaaggtgtctctcataccatacaaaggaggaatccattccaagcaaatctgttccgtcaagccgcagaacagctcgtgataggagaaaggataatgtgaggcagtctttaacttcagcggatccaacagcgctagtccaagaaattcgtttgcttgaaaaacatcaaaagaagttgggtgaagaagcaaaccaagcactggatctaattcacaaagaagtgacatcccataaactgggtgatcaacaagctgcagagaaagttgcgaaaatgctatcagaaatcagggacatgcagaagagcaaccttttaactgaggaaattgtagttggtgataaagctaacctgaaggaagaaataaatcgtttgaattctcaagaaattgcagctttggagaagaagctagagtgtgttcagaacactattgacattcttttcagacagatgaacagactccagatttcaggacccaggtgaaaaagaagagacttcttcccttcggcttaagtaatagtcctaacctacagcatatgatccggggaccatgttcacctctttctgggactgaaaataaggatcctgagagcaatgttgtgtctgctaattcaatcccagtgtcttttggagctactccaccaaagagggatgataaccgatgtcgtacaccgtcaagggaaggaacgcctgtatcacgacaagcaaattcagtcgatattaagagaatgaatcgaatgtataagaatgctgccgaagaaaatatacgtaacatcaaatcttatgttactggattgaaagaacgtgtggcgaagcttcaataccagaagcagcttcttgtttgccaggtcagtttacacttgtgacttatgctcattcatttacaaatttgttcatcaggaatcagtaacttaaaagcttgcacatgtataggtgttagaactggaggcaaacgagacaggcgctgcttcggagtacgatgcaaccgatgaatctcggatggactggccattgtgttttgaagagcagagaaagcaaatcataatgttatggcatctgtgccacatttcaatcattcacagaacacaattctacatgttattcaaaggagacccagctgatcaaatctacatggaagttgagctccgcaggctgacatggctcgaacagcacttggctgagcttgggaatgcgagtcctgcattgcttggtgatgaaccagcaagctacgtagcatcaaggtagtacaagagctttccttttcttcaattctatgagttaaaccatttttcacaagcatatatatgtctttggtgtgctgcagtataagagctttgaagcaggaaagagagtatttggcgaaacgggtaaacacaaagctaggagcagaggaaagagagatgctatacctgaaatgggatgtcccgccggtgggaaaacagaggaggcaacagttcatcaacaaactctggacagatcctcacaacatgcaacacgtgagagagagtgcggagatcgtggctaagcttgtcggattctgtgactcgggagagaccataaggaaagaaatgtttgagctcaactttgcatctccttcagataagaagacatggatgatgggttggaacttcatatccaacttgttgcatctctag

SEQ ID NO: 8 - DNA, 4570 bp, Artificial sequence; identical to SEQ ID NO: 6
  LER-R TES SEQUENCE

SEQ ID NO: 9 - DNA, 4570 bp, Artificial sequence
  TES-3 MUTANT SEQUENCE
atgggacctccgagaactccgttaagtaagatagataagtccaatccttatactccatgtggttccaaagttacagaggagaagattcttgtcactgttcggatgagaccattaaattggagggaacatgccaaatatgatctcattgcttgggaatgtcctgatgatgaaactatcgtctttaagaatccgaatcctgacaaggctccaacaaaatattcatttggtatttttattagaatctagtcaattgcttatttgtcttctcttctttccaattttgttatatagctcatttaagtttttgtcttgtttgtacttacagataaagtttttgagccaacttgtgccactcaagaggtgtatgaaggtggttctagggacgttgcactatctgcattagcaggaacaaatggtaaatacacagatatctaaaaaaatcccattatctaaggatattttatttgtggatatgtgatttcttgtctctatctattgcagcaactatttttgcttatggtcagactagtagtggcaagacgtttacaatgaggggggttacggaaagtgttgttaaagatatatacgaacatataagaaaagtgagtgaccaaatggatgtgttttggttttggttttgttacgaagtgatatgttctttttctatactcatggatctgttgaaatctatgtagactcaagaaaggagctttgtcttgaaagtatctgctttggagatctataatgagaccgtggttgaccttttaaatcgtgatactgggccccttcgactgttagatgatccagaggtaagtaaagggtcggaaacactaggctcatgaactatctcggccagtataagaatgttgtgtatgttgcagaaaggaaccattgtggaaaatctggtggaggaagtggtagaaagcaggcaacatttacagcatttaattagcatttgtgaaggtaattcttctatcacctgttaagatgctctatatctctttatgcatttgatgatgatagtattctaagtcatgtttagcaaagccttagaaccatgtagatgacatatataaaatatgactcattatttcactaatcactaatgagacatatgatatcatctttttatttcctgttatgtgattttccatttattgtacagatcaaaggcaagtaggtgaaactgctttaaacgacaaaagttcaagatcgcatcagattattcggctggtatgatctatactcatctgatatgctgcataattgaattaatattctattaaacatggagaagatagttttgcctagttatttgctaccattagacagtaaaagtaaccactttccataatttctcatgttctctgcttttaatattcagaccatccatagcagtctcagggaaatagcaggctgtgtgcagtctttcatggcaactctggtacgtcatttattatcatagggttgaagcttcacttggtattgacgacattttaattggtgcagaatcttgttgatcttgctggaagcgaacgtgcttttcaaacaaacgcagatggtctaagactgaaggaaggaagccacattaaccgtagtttattgacacttactacagtgatcagaaaactgaggtcagaaagcacactagtctgaacaccatgattatctttggcatgtctaaattaccttttaggttgatgaacattttcacccgtagggcaccttaagaaattagcaaaacatttaattatttctgttattttggtgaagttctggaagaaaaagggatcacgtgccatatagagactcaaagctgacaagaattttgcaaaattcacttggcgggaatgctagaacagcaattatctgtacgattagcccagcactgagtcacgttgaacagacaaaaaagacactctcttttgccatgagtgcgaaggaagtcaccaactgcgctaaagtaaacatggtatgtcacttagttaaatatttggagaggtctttcgttttgcagaaacaggatgattgtttggagctttgtctttggcttcctaaagcttttctttggtctttcattgaaaatgtggtttgtttgtgagtagcaatcatcatcctatagtgagcatatcatatgcaattatctgcctgcatagctattctgctcttcccatgaattaaggatacaaaatgcatcgcgttacctgtaatgatgtagtaagatataattcaaagccttctttgtcgctctcccactaacattactccttttgcttcaatcttctttgtgtttgatgaataacaaaacggttttgcttgttcttgatagaaagtttacccctctactggtattcaatttagggatgtgaatttagttgattttctgttgtaggttgtctcagaaaaaaagctgctgaaacatctgcagcaaaaagtagctaaactcgagtcagagttaagaagcccggagcccagttcatccacctgcttaaagtctctgttaattgagaaagagatgaaaatccaacaggtatgacttagactttaagaactagaattcacatcttgaattatctcttctgaatattttaaatgtggagtccacagactaggtaaaccatatgggacgtgaacttaagtttattaacaaaaacaacttgaattttttatgacttgcaattattcattcatgtttaactaatggcagatggaatccgaaatgaaagagctaaagcgtcagagagacattgcacagtctgaacttgatctagaaagaaaagcaaaggagcggaaggtatgtgcgctgctaatatgatataccaaaattttctgggataacttcaaccttatgcagacatgcctatataattggttgcagggatcaagtgagtgtgaacctttttctcaagtggcaaggtgtctctcataccatacaaaggaggaatccattccaagcaaatctgttccgtcaagccgcagaacagctcgtgataggagaaaggataatgtgaggcagtctttaacttcagcggatccaacagcgctagtccaagaaattcgtttgcttgaaaaacatcaaaagaagttgggtgaagaagcaaaccaagcactggatctaattcacaaagaagtgacatcccataaactgggtgatcaacaagctgcagagaaagttgcgaaaatgctatcagaaatcagggacatgcagaagagcaaccttttaactgaggaaattgtagttggtgataaagctaacctgaaggaagaaataaatcgtttgaattctcaagaaattgcagctttggagaagaagctagagtgtgttcagaacactattgacatgcttgtctcgtcttttcagacagatgaacagactccagatttcaggacccaggtgaaaaagaagagacttcttcccttcggcttaagtaatagtcctaacctacagcatatgatccggggaccatgttcacctctttctgggactgaaaataaggatcctgagagcaatgttgtgtctgctaattcaatcccagtgtcttttggagctactccaccaaagagggatgataactgatgtcgtacaccgtcaagggaaggaacgcctgtatcacgacaagcaaattcagtcgatattaagagaatgaatcgaatgtataagaatgctgccgaagaaaatatacgtaacatcaaatcttatgttactggattgaaagaacgtgtggcgaagcttcaataccagaagcagcttcttgtttgccaggtcagtttacacttgtgacttatgctcattcatttacaaatttgttcatcaggaatcagtaacttaaaagcttgcacatgtataggtgttagaactggaggcaaacgagacaggcgctgcttcggagtacgatgcaaccgatgaatctcggatggactggccattgtgttttgaagagcagagaaagcaaatcataatgttatggcatctgtgccacatttcaatcattcacagaacacaattctacatgttattcaaaggagacccagctgatcaaatctacatggaagttgagctccgcaggctgacatggctcgaacagcacttggctgagcttgggaatgcgagtcctgcattgcttggtgatgaaccagcaagctacgtagcatcaaggtagtacaagagctttccttttcttcaattctatgagttaaaccatttttcacaagcatatatatgtctttggtgtgctgcagtataagagctttgaagcaggaaagagagtatttggcgaaacgggtaaacacaaagctaggagcagaggaaagagagatgctatacctgaaatgggatgtcccgccggtgggaaaacagaggaggcaacagttcatcaacaaactctggacagatcctcacaacatgcaacacgtgagagagagtgcggagatcgtggctaagcttgtcggattctgtgactcgggagagaccataaggaaagaaatgtttgagctcaactttgcatctccttcagataagaagacatggatgatgggttggaacttcatatccaacttgttgcatctctag

SEQ ID NO: 10 - DNA, 4570 bp, Artificial sequence
  STUD- MUTANT SEQUENCE
atgggacctccgagaactccgttaagtaagatagataagtccaatccttatactccatgtggttccaaagttacagaggagaagattcttgtcactgttcggatgagaccattaaattggagggaacatgccaaatatgatctcattgcttgggaatgtcctgatgatgaaactatcgtctttaagaatccgaatcctgacaaggctccaacaaaatattcatttggtatttttattagaatctagtcaattgcttatttgtcttctcttctttccaattttgttatatagctcatttaagtttttgtcttgtttgtacttacagataaagtttttgagccaacttgtgccactcaagaggtgtatgaaggtggttctagggacgttgcactatctgcattagcaggaacaaatggtaaatacacagatatctaaaaaaatcccattatctaaggatattttatttgtggatatgtgatttcttgtctctatctattgcagcaactatttttgcttatggtcagactagtagtggcaagacgtttacaatgaggggggttacggaaagtgttgttaaagatatatacgaacatataagaaaagtgagtgaccaaatggatgtgttttggttttggttttgttacgaagtgatatgttctttttctatactcatggatctgttgaaatctatgtagactcaagaaaggagctttgtcttgaaagtatctgctttggagatctataatgagaccgtggttgaccttttaaatcgtgatactgggccccttcgactgttagatgatccagaggtaagtaaagggtcggaaacactaggctcatgaactatctcggccagtataagaatgttgtgtatgttgcagaaaggaaccattgtggaaaatctggtggaggaagtggtagaaagcaggcaacatttacagcatttaattagcatttgtgaaggtaattcttctatcacctgttaagatgctctatatctctttatgcatttgatgatgatagtattctaagtcatgtttagcaaagccttagaaccatgtagatgacatatataaaatatgactcattatttcactaatcactaatgagacatatgatatcatctttttatttcctgttatgtgattttccatttattgtacagatcaaaggcaagtaggtgaaactgctttaaacgacaaaagttcaagatcgcatcagattattcggctggtatgatctatactcatctgatatgctgcataattgaattaatattctattaaacatggagaagatagttttgcctagttatttgctaccattagacagtaaaagtaaccactttccataatttctcatgttctctgcttttaatattcagaccatccatagcagtctcagggaaatagcaggctgtgtgcagtctttcatggcaactctggtacgtcatttattatcatagggttgaagcttcacttggtattgacgacattttaattggtgcagaatcttgttgatcttgctggaagcgaacgtgcttttcaaacaaacgcagatggtctaagactgaaggaaggaagccacattaaccgtagtttattgacacttactacagtgatcagaaaactgaggtcagaaagcacactagtctgaacaccatgattatctttggcatgtctaaattaccttttaggttgatgaacattttcacccgtagggcaccttaagaaattagcaaaacatttaattatttctgttattttggtgaagttctggaagaaaaagggatcacgtgccatatagagactcaaagctgacaagaattttgcaaaattcacttggcgggaatgctagaacagcaattatctgtacgattagcccagcactgagtcacgttgaacagacaaaaaagacactctcttttgccatgagtgcgaaggaagtcaccaactgcgctaaagtaaacatggtatgtcacttagttaaatatttggagaggtctttcgttttgcagaaacaggatgattgtttggagctttgtctttggcttcctaaagcttttctttggtctttcattgaaaatgtggtttgtttgtgagtagcaatcatcatcctatagtgagcatatcatatgcaattatctgcctgcatagctattctgctcttcccatgaattaaggatacaaaatgcatcgcgttacctgtaatgatgtagtaagatataattcaaagccttctttgtcgctctcccactaacattactccttttgcttcaatcttctttgtgtttgatgaataacaaaacggttttgcttgttcttgatagaaagtttacccctctactggtattcaatttagggatgtgaatttagttgattttctgttgtaggttgtctcagaaaaaaagctgctgaaacatctgcagcaaaaagtagctaaactcgagtcagagttaagaagcccggagcccagttcatccacctgcttaaagtctctgttaattgagaaagagatgaaaatccaacaggtatgacttagactttaagaactagaattcacatcttgaattatctcttctgaatattttaaatgtggagtccacagactaggtaaaccatatgggacgtgaacttaagtttattaacaaaaacaacttgaattttttatgacttgcaattattcattcatgtttaactaatggcagatggaatccgaaatgaaagagctaaagcgtcagagagacattgcacagtctgaacttgatctagaaagaaaagcaaaggagcggaaggtatgtgcgctgctaatatgatataccaaaattttctgggataacttcaaccttatgcagacatgcctatataattggttgcagggatcaagtgagtgtgaacctttttctcaagtggcaaggtgtctctcataccatacaaaggaggaatccattccaagcaaatctgttccgtcaagccgcagaacagctcgtgataggagaaaggataatgtgaggcagtctttaacttcagcggatccaacagcgctagtccaagaaattcgtttgcttgaaaaacatcaaaagaagttgggtgaagaagcaaaccaagcactggatctaattcacaaagaagtgacatcccataaactgggtgatcaacaagctgcagagaaagttgcgaaaatgctatcagaaatcagggacatgcagaagagcaaccttttaactgaggaaattgtagttggtgataaagctaacctgaaggaagaaataaatcgtttgaattcttaagaaattgcagctttggagaagaagctagagtgtgttcagaacactattgacatgcttgtctcgtcttttcagacagatgaacagactccagatttcaggacccaggtgaaaaagaagagacttcttcccttcggcttaagtaatagtcctaacctacagcatatgatccggggaccatgttcacctctttctgggactgaaaataaggatcctgagagcaatgttgtgtctgctaattcaatcccagtgtcttttggagctactccaccaaagagggatgataaccgatgtcgtacaccgtcaagggaaggaacgcctgtatcacgacaagcaaattcagtcgatattaagagaatgaatcgaatgtataagaatgctgccgaagaaaatatacgtaacatcaaatcttatgttactggattgaaagaacgtgtggcgaagcttcaataccagaagcagcttcttgtttgccaggtcagtttacacttgtgacttatgctcattcatttacaaatttgttcatcaggaatcagtaacttaaaagcttgcacatgtataggtgttagaactggaggcaaacgagacaggcgctgcttcggagtacgatgcaaccgatgaatctcggatggactggccattgtgttttgaagagcagagaaagcaaatcataatgttatggcatctgtgccacatttcaatcattcacagaacacaattctacatgttattcaaaggagacccagctgatcaaatctacatggaagttgagctccgcaggctgacatggctcgaacagcacttggctgagcttgggaatgcgagtcctgcattgcttggtgatgaaccagcaagctacgtagcatcaaggtagtacaagagctttccttttcttcaattctatgagttaaaccatttttcacaagcatatatatgtctttggtgtgctgcagtataagagctttgaagcaggaaagagagtatttggcgaaacgggtaaacacaaagctaggagcagaggaaagagagatgctatacctgaaatgggatgtcccgccggtgggaaaacagaggaggcaacagttcatcaacaaactctggacagatcctcacaacatgcaacacgtgagagagagtgcggagatcgtggctaagcttgtcggattctgtgactcgggagagaccataaggaaagaaatgtttgagctcaactttgcatctccttcagataagaagacatggatgatgggttggaacttcatatccaacttgttgcatctctag